(19) United States
(12) Patent Application Publication
(10) Pub. No.: US 2012/0245147 A1
(43) Pub. Date: Sep. 27, 2012

(54) INHIBITORS OF THAPSIGARGIN-INDUCED CELL DEATH

(76) Inventors: John C. REED, Rancho Santa Fe, CA (US); In-Ki Kim, San Diego, CA (US)

(21) Appl. No.: 13/451,187

(22) Filed: Apr. 19, 2012

Related U.S. Application Data
(62) Division of application No. 12/154,544, filed on May 23, 2008, now abandoned.
(60) Provisional application No. 60/931,969, filed on May 25, 2007.

(51) Int. Cl. A61K 31/5513; C12N 5/0797; A61K 31/495; A61K 31/496; A61K 31/55; A61K 31/4709; A61K 31/404; A61K 31/429; A61K 31/517; A61P 25/00; A61P 3/00; A61P 9/10; A61P 9/00; A61P 31/12; A61P 25/28; A61P 25/16; A61P 3/10; A61P 3/06; C12N 5/071

(52) U.S. Cl. 514/217.08; 435/375; 435/353; 514/220; 514/255.01; 514/254.08; 514/313; 514/418; 514/368; 514/266.31

(57) ABSTRACT

Methods for screening for inhibitors of endoplasmic reticulum (ER) stress are provided. These methods involve the addition of thapsigargin, which induces ER stress, and a test agent to mammalian cells in multi-well plates. Cell survival can be readily monitored by measuring intracellular ATP content using a bioluminescent reagent. Screening a commercially available library of 50,000 compounds led to the identification of 93 hit compounds that were subjected to secondary assays to confirm their ability to rescue cells from thapsigargin-induced cell death.

### CROSS-REFERENCE TO RELATED CASES

This application claims priority from U.S. provisional patent application Ser. No. 60/931,969, filed 25 May 2007, which is incorporated herein by reference in its entirety.

### STATEMENT OF GOVERNMENT RIGHTS

This invention was made with Government support under RO3 DA024887 and U01 AI078048 awarded by the National Institutes of Health. The Government has certain rights in the invention.

### TECHNICAL FIELD

The present invention relates to inhibitors of cell death caused by the unfolded protein response.

### BACKGROUND

The endoplasmic reticulum (ER) fulfills multiple cellular functions (reviewed in Schroder and Kaufman, Mutat. Res., 569:29-63, 2005; Shen et al., J. Chem. Neuroanat. 28:79-92, 2004; Rao et al., Cell Death Differ. 11:372-380, 2004; Breckenridge et al., Oncogene 22:8608-8618, 2003). The lumen of the ER is a unique environment. It contains the highest concentration of Ca²⁺ within the cell due to the active transport into the ER of calcium ions by Ca²⁺-ATPases. The lumen possesses an oxidative environment, critical for formation of disulfide-bonds and proper folding of proteins destined for secretion or display on the cell surface. Because of its role in protein folding and transport, the ER is also rich in Ca²⁺-dependent molecular chaperones, such as Grp78, Grp94, and calreticulin, which help stabilize protein folding intermediates (reviewed in (Schroder and Kaufman, Mutat. Res. 569:29-63, 2005; Orrenius et al., Nat. Rev. Mol. Cell Biol. 4:552-565, 2003; Ma and Hendershot, J. Chem. Neuroanat. 28:51-65, 2004; Rizzuto et al., Sci. STKE, 2004: re1, 2004).

Myriad types of disturbances cause accumulation of unfolded proteins in the ER, triggering an evolutionarily conserved response, termed the unfolded protein response (UPR). Disturbances in cellular redox regulation, caused by hypoxia, oxidants, or reducing agents, interfere with disulfide bonding in the lumen of the ER, leading to protein unfolding and misfolding (Frand et al., Trends Cell Biol. 10:203-210, 2000). Glucose deprivation also leads to ER stress, probably by interfering with N-linked protein glycosylation in the ER. Aberrations of Ca²⁺ regulation in the ER cause protein unfolding, because of the Ca²⁺-dependent nature of ER proteins, Grp78, Grp94, and calreticulin (Ma and Hendershot, J. Chem. Neuroanat. 28:51-65, 2004). Viral infection may also trigger the UPR, due to the overload of the ER with virus-encoded proteins, possibly representing one of the ancient evolutionary pressures for linking ER stress to cell suicide for avoiding replication and spread of viruses. Also, because a certain amount of basal protein misfolding occurs in the ER, normally ameliorated by retrograde transport of misfolded proteins into the cytosol for proteasome-dependent degradation, situations that impair proteasome function can create a veritable protein traffic jam, including inclusion body diseases associated with neurodegeneration (Paschen, Cell Calcium 34:365-383, 2003). High fat diets have also recently been associated with triggering ER stress (Ozcan et al., Science 306:457-461, 2004).

The initial purpose of the UPR is to adapt to the changing environment, and reestablish homeostasis and normal ER function. These adaptive mechanisms predominantly involve activation of transcriptional programs that induce expression of genes that enhance the protein folding capacity of the ER, and promote ER-associated protein degradation to remove misfolded proteins. Translation of mRNAs is also initially inhibited, thereby reducing the influx of new proteins into the ER, for a few hours until mRNAs encoding UPR proteins are produced. When adaptation fails, ER-initiated pathways signal alarm by activating NFκB, a transcription factor that induces expression of genes encoding mediators of in host-defense, and activation of stress kinases (p38 MAPK and JNK). Excessive and prolonged ER stress triggers cell suicide, usually in the form of apoptosis in animal cells, representing a last resort of multicellular organisms to dispense of dysfunctional cells. ER stress has been associated with a wide range of diseases, including ischemia-reperfusion injury (particularly stroke), neurodegeneration, and diabetes (reviewed in (Oyadomari and Mori, Cell Death Differ. 11:381-389, 2004; Xu et al., J. Clinical Invest. 115:2656-2664, 2005; Rao and Bredesen, Curr. Opin. Cell Biol. 16:653-662, 2004).

When unfolded proteins accumulate in the ER, resident chaperones become occupied, releasing transmembrane ER proteins involved in inducing the UPR. These UPR-initiating proteins straddle ER membranes, with their N-terminus in the lumen of the ER and their C-terminus in the cytosol, providing a bridge that connects these two cellular compartments. Normally, the N-termini of these transmembrane ER proteins are held by ER charperone Grp78 (BiP), preventing their aggregation. But, when misfolded proteins accumulate, Grp78 releases, allowing aggregation of these transmembrane signaling proteins, and launching the UPR. Among the critical transmembrane ER signaling proteins are PERK, Ire1, and ATF6 (FIG. 1) (reviewed in Schroder and Kaufman, Mutat. Res. 569:29-63, 2005; Shen et al., J. Chem. Neuroanat. 28:79-92, 2004; Xu et al., J. Clinical Invest. 115:2656-2664, 2005; Rao and Bredesen, Curr. Opin. Cell Biol. 16:653-662, 2004).

PERK (PKR-like ER Kinase) is a Ser/Thr-protein kinase, the catalytic domain of which shares substantial homology to other eIF2α-family kinases (Shi et al., Mol. Cell Biol. 18:7499-7509, 1998; Harding et al., Nature 397:271-274, 1999). Upon removal of Grp78, PERK oligomerizes in ER membranes, thereby inducing its autophosphorylation and activating the kinase domain. PERK phosphorylates and inactivates the eukaryotic translation initiation factor 2 alpha (eIF2α), thereby globally shutting off mRNA translation and reducing the protein load on the ER. However, certain mRNAs gain a selective advantage for translation under these conditions, including the mRNA encoding transcription factor ATF4. The 39 kDa ATF4 protein is a member of the bZIP-family of transcription factors, which regulates the promoters of several genes implicated in the UPR. The importance of PERK-initiated signals for protection against ER stress has been documented by studies of perk−/− cells and of knock-in cells that express nonphosphorylatable eIF2α(Ser51Ala), both of which display hypersensitivity to ER stress (Harding et al., Mol. Cell, 5:897-904, 2000; Scheuner et al., Mol. Cell 7:1165-1176, 2001). Ire1 similarly oligomerizes in ER membranes when released by Grp78. The ˜100 kDa Ire1α protein is a type I transmembrane protein, which contains both a Ser/Thr-kinase domain and an endoribonuclease domain, the latter which processes an intron from X box-binding protein-1 (XBP-1) mRNA, rendering it competent for translation to produce the 41 kDa XBP-1 protein, a bZIP-family transcription factor. XBP-1 binds to promoters of several genes involved predominantly in retrograde transport of misfolded proteins from ER to cytosol and in ER-induced protein degradation (reviewed in Rao and Bredesen, Curr. Opin. Cell Biol. 16:653-662, 2004). Ire1 also shares in common with many members of the Tumor Necrosis Factor (TNF) receptor family the ability to bind adapter protein TRAF2.

TRAF2 is an E3 ligase that binds Ubc13, resulting in non-canonical polyubiquitination of substrates involving lysine 63 rather than the canonical lysine 48 as a linking site (Habelhah et al., EMBO J. 23:322-332, 2004). TRAF2 activates protein kinases previously implicated in immunity and inflammation, including Ask1, which activates Jun-N-terminal kinase (JNK), and kinases linked to NFκB activation. Release of Grp78 from the N-terminus of ATF6 triggers a different mechanism of protein activation, compared to PERK and Ire1. Instead of oligomerizing, release of Grp78 frees ATF6 to translocate to the Golgi, where resident proteases cleave ATF6 at a juxtamembrane site, releasing this transcription factor into the cytosol and allowing it to migrate into the nucleus to regulate gene expression (Ye et al., Mol. Cell 6:1355-1364, 2000).

How these various signaling pathways induced by ER stress trigger cell death is unclear. This is the subject of a recent review we authored where the many possibilities were outlined (Xu et al., J. Clinical Invest. 115:2656-2664, 2005). Compounds that block cell death induced specifically as a result of ER stress (and not other cell death pathways) would be useful for interrogating the underlying mechanisms, as well as for ascertaining in vivo in animal models when ER stress is the inciting event responsible for cell demise and tissue injury.

### SUMMARY OF THE INVENTION

We have developed novel high-throughput methods for screening for inhibitors of endoplasmic reticulum (ER) stress. These methods involve the addition of thapsigargin, which induces ER stress, and a test agent to mammalian cells in multi-well plates. Cell survival can be readily monitored by measuring intracellular ATP content using a bioluminescent reagent. Screening a commercially available library of 50,000 compounds led to the identification of 93 hit compounds that were subjected to secondary assays to confirm their ability to rescue cells from thapsigargin-induced cell death.

According to one embodiment of the invention, methods are provided to identify an inhibitor of cell death resulting from endoplasmic reticulum stress, comprising: (a) contacting a mammalian cell with thapsigargin, thereby causing endoplasmic reticulum stress in the cell; (b) contacting the cell with a test agent; and (c) determining whether the test agent inhibits death of the cell caused by endoplasmic reticulum stress. According to one such embodiment, the mammalian cell is a CSM14.1 rat striatal neuroprogenitor cell. According to another such embodiment, the method further comprises determining whether the test agent inhibits death of the cell caused by endoplasmic reticulum stress by measuring intracellular ATP content of the cell. According to another such embodiment, the method further comprises measuring intracellular ATP content of the cell by measuring bioluminescence of the cell. According to another such embodiment, the method comprises determining whether the test agent inhibits death of the cell by about 50% or more, or about 60% or more, or about 70% or more, or about 80% or more, or about 90% or more, or about 95% or more. According to another such embodiment, the method comprises determining whether the test agent has an IC₅₀ of about 25 μM or less, or about 20 μM or less, or about 15 μM or less, or about 10 μM or less. According to another such embodiment, the method comprises contacting the cell with the test agent after contacting the cell with thapsigargin. According to another such embodiment, the method comprises providing the cell in a well of a multi-well plate. According to another such embodiment, the method is automated.

According to another embodiment, compositions are provided that comprise an effective amount of a compound that inhibits death of a mammalian cell resulting from endoplasmic reticulum stress induced by thapsigargin. According to one such embodiment, the mammalian cell is a CSM14.1 rat striatal neuroprogenitor cell. According to another such embodiment, such a composition inhibits death of CSM14.1 rat striatal neuroprogenitor cells by about 50 percent or more, or 60 percent or more, or 70 percent or more, or 80 percent or more, or 90 percent or more, or 95 percent or more. According to another such embodiment, the composition has an IC₅₀ of about 25 μM or less, or about 20 μM or less, or about 15 μM or less. According to another such embodiment, the composition inhibits death of CSM14.1 rat striatal neuroprogenitor cells by about 50 percent or more and has an IC₅₀ of about 25 μM or less.

According to another such embodiment, the composition comprises a compound selected from the group consisting of ChemBridge ID numbers 5230707, 5397372, 5667681, 5706532, 5803884, 5843873, 5850970, 5897027, 5923481, 5926377, 5931335, 5933690, 5947252, 5948365, 5951613, 5954179, 5954693, 5954754, 5955734, 5962263, 5963958, 5974219, 5974554, 5976228, 5979207, 5980750, 5981269, 5984821, 5986994, 5990041, 5990137, 5993048, 5998734, 6000398, 6015090, 6033352, 6034397, 6034674, 6035098, 6035728, 6037360, 6038391, 6043815, 6044350, 6044525, 6044626, 6044673, 6044860, 6045012, 6046070, 6046818, 6048306, 6048935, 6049010, 6049184, 6049448, 6056592, 6060848, 6062505, 6065757, 6066936, 6068189, 6068602, 6069474, 6070379, 6073875, 6074259, 6074532, 6074891, 6081028, 6084652, 6094957, 6095577, 6095970, 6103983, 6104939, 6141576, 6237735, 6237877, 6237973, 6237992, 6238190, 6238246, 6238475, 6238767, 6239048, 6239252, 6239507, 6239538, 6239939, 6241376, 6368931, and 6370710. According to another such embodiment, the composition comprises a compound of Formula I, including but not limited to ChemBridge ID numbers 6239507, 6237735, 6238475, 6237877, 6239538, 6238767, 6049448, 5963958, 6237973, and 6044673. According to another such embodiment, the composition comprises a compound of Formula II-1, including but not limited to ChemBridge ID numbers 5998734, 5955734, 5990041, 6035098, and 5990137. According to another such embodiment, the composition comprises a compound of Formula II-2, including but not limited to ChemBridge ID numbers 5397372, 6033352, 6034674, and 5951613. According to another such embodiment, the composition comprises a compound selected from the group consisting of ChemBridge ID numbers 5948365, 5976228, 5980750, 5803884, 6049184, 5979207, and 6141576. According to another such embodiment, the composition comprises a pharmaceutically acceptable carrier.

According to another embodiment, kits are provided that comprise (a) one of the aforementioned compositions and (2) suitable packaging.

According to another embodiment, methods are provided for inhibiting death of a mammalian cell resulting from endoplasmic reticulum stress comprising treating the cell with any of the aforementioned compositions.

According to another embodiment, methods are provided for treating a disease, condition or injury of a mammal (including but not limited to a human) associated with endoplasmic reticulum stress comprising administering to a mammal in need thereof any of the aforementioned compositions. According to one such embodiment, the disease, condition or injury is selected from the group consisting of neuronal disease, metabolic disease, ischemia injury, heart and circulatory system injury, viral infection; atherosclerosis, bipolar disease, and Batten disease. According to another such embodiment, the neuronal disease is selected from the group consisting of familial Alzheimer's disease, Parkinson disease, Huntington disease, spinobulbar muscular atrophy/Kennedy disease, spinocerebellar ataxia 3/Machado-Joseph disease, prion disease, amyotrophic lateral sclerosis, and GM1 gangliodosis. According to another such embodiment, the metabolic disease is selected from the group consisting of diabetes mellitus general, Wolcott-Rallison syndrome, Wolfran syndrome, type 2 diabetes mellitus, homocysteinemia, Zα1-antitrypsin deficiency inclusion body myopathy, and hereditary tyrosinemia type 1. According to another such embodiment, the heart and circulatory system injury is selected from the group consisting of cardiac hypertrophy, hypoxic damage, and familial hypercholesterolemia.

According to another embodiment, the invention provides the use of an ER stress inhibitory compound to prepare a medicament for administration to an individual in need thereof.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE FIGURES

FIG. 1 shows the structure of hit compounds from Group 1 and Formula I, based on the compounds of Group 1.

FIG. 2A shows the structure of hit compounds from Group 2.

FIG. 2B shows Formula 2-1 (based on the compounds of Group 2-1) and Formula 2-2 (based on the compounds of group 2-2).

FIG. 3 shows the structure of five independent hit compounds that do not fall into Groups 1 or 2.

FIG. 4 shows the results of pilot studies for use of CSM14.1 neuronal cells for studying ER stress-induced cell death. (A) Evaluation of cell density. (B) Dose-response for thapsigargin (TG). (C) Dose response for Salubrinal (Sal).

FIG. 5 shows that TG kills and Sal protects undifferentiated (FIG. 5A) and differentiated (FIG. 5B) CSM14.1 cells.

FIG. 6 shows an assessment of the reproducibility of the ATP content assay.

FIG. 7 shows an assay quality control analysis.

FIG. 8 shows a flow chart from screening to hit compound identification.

FIG. 9 shows raw data analysis results (A) and normalized relative survival rate calculations (B) from a typical screening of an in-house library of 50,000 compounds showing one efficient hit compound (bold) at column 8, row G, corresponding to a survival rate of 98.9%.

FIG. 10 shows a graphical representation of an example of screening results after normalization of data. Relative ATP content (y-axis) is plotted against well number (1-96 [A1 to H12]) (x-axis).

FIG. 11 shows the dose-dependent inhibition of ER stress-induced cell death by hit compounds. Undifferentiated CSM 14.1 cells were treated with thapsigargin (15 μM) and with various concentrations of four of the hit compounds (A, B, C, D). Cellular ATP levels were measured (y-axis) and plotted against compound concentration (x-axis). The data are representative of three independent experiments.

FIG. 12 shows that salubrinal inhibits thapsigargin-induced cell death less efficiently than our hit compounds.

FIG. 13 shows that our hit compounds inhibit tunicamycin-induced cell death with an efficiency that is comparable with salubrinal.

FIG. 14 shows a comparison of the cytoprotective activity of compounds using undifferentiated versus differentiated CSM14. neuronal cells. White bars=DMSO controls; Black bars=compounds that protect both differentiated and undifferentiated cells; Gray bars=compounds that protect undifferentiated but not differentiated neurons.

FIG. 15 shows cell-type specificity of compounds in protecting against ER stress. CSM 14.1 (left) and Jurkat (right) cells were cultured overnight at 3,000 cells per well or at 30,000 cells per well, respectively, in 96-well plates. Wells received DMSO alone (white bars) or 25 μM compounds (A-C) in DMSO, followed by treatment with (+) or without (−) TG (15 μM). After culturing for 24 hrs, ATP content was determined, expressing data as a percentage control relative to cells treated only with DMSO (mean±SD; n=3).

FIG. 16 shows the results of a secondary assay for evaluating the cytoprotective activity of compounds. Undifferentiated CSM14.1 cells were cultured at 10⁴ cells per well of 24-well plates. The next day, DMSO (a, b) (1% final volume), 100 μM Salubrinal (c, d) or 25 μM of hit compounds (1% final DMSO) was added. After two hrs, 15 μM TG was added to all wells except a and c. A conventional ATP assay was performed to measure survival rate. Treatment: a: 1% DMSO; b: 1% DMSO, 15 μM TG; c: 100 μM Sal; d: 100 μM Sal, 15 μM TG; Compounds (ChemBridge Compound ID):

| Number | Compound |
|---|---|
| 1 | 6239507 |
| 2 | 6237735 |
| 3 | 5998734 |
| 4 | 6238475 |
| 5 | 6237877 |
| 6 | 5397372 |
| 7 | 6239538 |
| 8 | 5955734 |
| 9 | 6238767 |
| 10 | 6049448 |
| 11 | 5990041 |
| 12 | 5976228 |
| 13 | 5963958 |
| 14 | 5979207 |
| 15 | 5980750 |
| 16 | 5803884 |
| 17 | 6033352 |
| 18 | 6237973 |
| 19 | 6141576 |
| 20 | 5951613 |
| 21 | 6044673 |
| 22 | 5948365 |
| 23 | 6034674 |
| 24 | 6035098 |
| 25 | 5990137 |
| 26 | 6049184 |

FIG. 17 shows the results of a secondary assay for evaluating the cytoprotective activity of compounds. Undifferentiated CSM14.1 cells were cultured as for FIG. 16. The next day, DMSO (a, b) (1% final volume), 100 μM Salubrinal (c, d) or 25 μM of hit compounds (1% final DMSO) was added. After two hrs, 15 μM TG was added to all wells except a and c. The plates were returned to culture for 24 hrs, then cells were recovered by trypsinization, transferred to 1.5 ml microcentrifuge tubes, and resuspended in 0.5 mL of Annexin V-binding solution. The percentage of annexin V-negative cells was determined by flow-cytometry (y-axis). Treatments and compounds were the same as in FIG. 16.

FIG. 18 shows the pathway selectivity of the hit compounds. Undifferentiated CSM 14.1 cells were plated at 3,000 cells per well in 96-well plates (for ATP assay) or at 1×10⁴ cells per well in 24-well plates (for flow cytometry). The next day, cells were treated with DMSO (0.5%) or hit compounds 25 μM of a compound with 0.5% DMSO final concentration) for two hours, followed by treatment with various cell death-inducing reagents, including 15 μM thapsigargin (TG) for 24 hrs, 10 μg/mL tunicamycin (TU) for 72 hrs, 2.5 μM staurosporine (STS) for 24 hrs, 50 μM VP16 for 48 hrs, or 30 ng/mL TNF plus 10 μg/mL cyclohexamide (CHX) for 24 hrs. Cellular ATP content was measured for staurosporine samples and TNF/CHX samples, normalizing data relative to cells treated with DMSO alone (the control) and presenting as a percentage of control. For measuring cell death resulting from treatment with tunicamycin and VP16, flow cytometry was used. All assays were performed in triplicate (mean±SD).

FIG. 19 shows that ER stress inhibitory compounds inhibit TG-induced markers of Ire1 pathway. CSM 14.1 cells were cultured with DMSO or with 25 μM of hit compounds for two hours, followed by treatment of thapsigargin (15 μM). Cell lysates were prepared and analyzed by SDS-PAGE/immunoblotting using antibodies specific for phospho-c-Jun, phospho-eIF2α, phospho-p38 MAPK, and tubulin (loading control). Controls lanes were treated with DMSO alone or DMSO plus TG. In another experiment, CSM14.1 cells were cultured with either DMSO or one of the active compounds at 1, 5, and 10 μM, followed two hours later by 15 μM TG. After two hrs, cell lysates were prepared, normalized for protein content, and either analyzed by SDS-PAGE/immunoblotting using anti-p38-MAPK pan-reactive antibody or phospho-specific antibody with ECL-based detection, followed by densitometry analysis of x-ray films, normalizing phosphor-p38 MAPK relative to total p38 MAPK, or analyzed using a meso-scale instrument from MSD and a procedure in which total p38 MAPK is captured on plates, and the relative amounts of phosphorylated protein are determined using phospho-specific antibody (MSD catalog #K15112D1).

FIG. 20 shows a route for resynthesis of CID-2878746 and synthesis of MLS-0292126.

FIG. 21 shows the unfolded protein response (UPR) signal transduction pathways.

FIG. 22 shows the results of in vitro kinase assays using compound 6239507.

FIG. 23 shows that phosphorylation of the ser 967 site of ASK1 was intensified by compound 6239507, which inhibits ER stress. Phosphorylation of ASK1 at various sites was inspected. 293T cells were transfected with pcDNA-ASK1-HA. One day later, cells were incubated with DMSO (0.4%) or 100 μM compound 6239507 (#1) for two hours. Cell extracts were prepared using lysis buffer and were subjected to immunoblotting using anti-phospho ASK1 antibodies or anti HA antibody as indicated (A). The relative density of each phosphorylated ASK band was calculated by imageJ software (B). In (C), compounds from (A) were compared in activity against thapsigargin-induced cell death. For the negative control, hit compound #14 was used (left gray bar); compound #14 is a potent inhibitor of cell death but has a different structure than compound 6239507. As another negative control, compound 6048163 was used (right gray bar); it shares the same backbone as the hit compounds but is not potent as an inhibitor of cell death. (D) 293T cells were transfected with pcDNA-ASK1-HA and pEBG-GST-14-3-3. One day later cells were incubated with DMSO (0.4%) or 100 μM of the indicated compound for two hours. Then cells were treated with thapsigargin (20 μM) for the indicated time. Cell extracts were prepared using lysis buffer, and 14-3-3 proteins were immunoprecipitated with glutathione S transferase 4B sepharose beads. ASK1 protein binding with 14-3-3 was visualized by immunoblotting using anti-HA antibody. Anti-phospho ASK1 (ser967) antibody was used to detect phosphorylation of ASK1 at each time point.

FIG. 24 shows our hypothesis that the hit benzodiazepine compounds are inhibitors of ASK1 ser967 dephosphorylation. Thus, the compounds inhibit dissociation of 14-3-3 from ASK1, rendering ASK1 inactive.

FIG. 25 shows that compound 6239507 can inhibit ER stress-induced cell death in primary mouse neuronal cells. Primary cortical neuron cells were prepared from the midbrain of mice. After 14 days of maturation, the cells were preincubated with DMSO (0.2%) or 25 μM of compound 6239507 for two hours. The cells were then treated with thapsigargin (TG) for 24 hours. Cells were fixed with an aldehyde solution and subjected to immunostaining with NeuN and MAP2 antibody for staining the neuronal body and axon network. Hoechst dye was used to stain nuclei. To show the loss of the axon network by thapsigargin, a wide field was captured by fluorescent microscopy. Cells showing a condensed nucleus and shrunken neuritis were considered as dead to evaluate cell death.

FIG. 26 shows relative survival for CSM14.1 cells treated with various hit compounds. CSM14.1 cells were plated at 1,500 cells per well in 96-well plates and cultured at 39° C. (non-permissive temperature) for 7 days. Hit compounds were added to a final concentration of 25 μM followed two hour later by thapsigargin (TG) at a final concentration of 15 μM. ATP content was measured and data were expressed as a percentage of control cells treated with only 1% DMSO (mean±SD; n=3).

FIG. 27 shows that ER stress inhibitory compounds inhibit thapsigargin-induced markers of the Ire1 pathway. CSM 14.1 cells were cultured with DMSO or with the indicated compounds at 1 μM, 5 μM, and 10 μM, followed by treatment with thapsigargin (15 μM). After two hours, cell lysates were prepared, normalized for protein content, and either analyzed by SDS-PAGE/immunoblotting using anti-p38 MAPK pan-reactive antibody or phosphor-specific antibody with ECL-based detection (top), followed by densitometry analysis of x-ray films, normalizing phospho-p38 MAPK relative to total p38 MAPK (middle), or analyzed using a meso-scale instrument from MSD and a procedure in which total p38 MAPK is captured on plates, and the relative amounts of phosphorylated protein are determined suing phosphor-specific antibody (MSD catalog #K15112D1) (bottom).

### DETAILED DESCRIPTION OF THE INVENTION

The present invention provides a method for screening compounds that inhibit ER stress, compounds that are identified using such a screen, and related compositions and methods.

### DEFINITIONS

As used herein, “ER stress inhibitory compound” refers to a compound that has “ER stress inhibitory activity,” namely, that inhibits cell death resulting from ER stress, preferably by about 50% or more, or 60% or more, or 70% or more, or 80% or more, or 90% or more, as measured by a suitable assay. Preferably, the ER stress inhibitory compound is effective in treating any disease, disorder, condition or injury associated with ER stress. Preferably the ER stress inhibitory compound has an IC₅₀ of about 25 μM or less, or 20 μM or less, or 15 μM or less, or 10 μM or less. In a high-throughput screen of a library of 50,000 compounds, we identified 93 ER stress inhibitory compounds (“hits”) that inhibited cell death due to ER stress resulting from thapsigargin treatment. Of these 93 hits, 30 were determined to have an IC₅₀ of 25 μM or less. The ER stress inhibitory compounds of the invention also include pharmaceutically acceptable analogs, prodrugs, salts or solvates of any of the ER stress inhibitory compounds provided herein. Also included are compounds that are structurally related to any of the ER stress inhibitor compounds provided herein and that have ER stress inhibitory activity, including but not limited to compounds listed in Tables 3 and 6-11.

(Herein, compounds having a particular ChemBridge Compound ID number, may simply be referred to as “compound <number>” or even by number alone. For example, ChemBridge Compound ID 5230707 may be referred to as “compound 5230707” or “5230707”. Additional information about individual compounds, including their chemical structure, chemical name, molecular weight, etc., are available for each compound at the ChemBridge Corporation website: www.hit2lead.com.

ER stress inhibitory compounds include but are not limited to the compounds listed in Table 1 below, which protect CSM14.1 cells from thapsigargin-induced cell death.

TABLE 1
List of hit compounds that protect CSM14.1
cells from thapsigargin-induced cell death.

| | Source | | | |
|---|---|---|---|---|
| ChemBridge | Plate | Coordi- | | Mol. |
| Compound ID | ID | nates | Formula | Wt. |
| 5230707 | 10809 | H2 | C₂₇H₂₉ClN₂O | 423.98 |
| 5397372 | 10877 | G2 | C₂₉H₃₃N₃O₄ | 487.59 |
| 5667681 | 10968 | H5 | C₂₃H₂₅BrN₈O₄ | 557.4 |
| 5706532 | 10987 | B3 | C₂₉H₂₇N₃O₂ | 449.55 |
| 5803884 | 11053 | B5 | C₂₃H₂₂N₂O₂ | 358.43 |
| 5843873 | 11003 | A8 | C₁₉H₁₆N₂O₄ | 336.34 |
| 5850970 | 11067 | D7 | C₂₅H₂₇ClN₂O₂ | 422.95 |
| 5897027 | 11078 | H4 | C₂₆H₂₅FN₂O₅ | 464.49 |
| 5923481 | 11091 | A6 | C₂₅H₂₅N₃O₄ | 431.49 |
| 5926377 | 11097 | A7 | C₂₁H₂₃N₃O₂ | 349.43 |
| 5931335 | 11102 | G2 | C₂₀H₂₀N₄OS•BrH | 445.38 |
| 5933690 | 11102 | F10 | C₂₂H₂₁N₃O₃ | 375.42 |
| 5947252 | 11106 | D8 | C₁₉H₁₆N₂O₂ | 304.34 |
| 5948365 | 11089 | A8 | C₂₅H₂₅N₃O₃•C₂H₂O₄ | 505.52 |
| 5951613 | 11246 | B4 | C₂₄H₂₅N₃OS•C₂H₂O₄ | 493.58 |
| 5954179 | 11200 | E3 | C₁₈H₁₂F₃N₃O | 343.3 |
| 5954693 | 11033 | G7 | C₂₆H₂₇N₃O₅ | 461.51 |
| 5954754 | 11104 | G5 | C₂₆H₂₈N₂O | 384.52 |
| 5955734 | 11105 | F2 | C₂₆H₂₈N₂O₃ | 416.51 |
| 5962263 | 11185 | C3 | C₁₂H₉NOS₂ | 247.34 |
| 5963958 | 10796 | D2 | C₂₆H₂₄N₂OS | 412.55 |
| 5974219 | 11115 | G6 | C₃₁H₂₅N₃O₂ | 471.55 |
| 5974554 | 11115 | F8 | C₂₉H₂₉FN₂O₃ | 472.55 |
| 5976228 | 10800 | D8 | C₂₇H₂₆N₂O | 394.51 |
| 5979207 | 11121 | E5 | C₁₇H₁₅N₂OS₂•Br | 407.35 |
| 5980750 | 11121 | D4 | C₂₆H₂₇N₃O₄S | 477.58 |
| 5981269 | 11116 | C10 | C₂₂H₁₈N₂O | 326.39 |
| 5984821 | 11123 | E9 | C₂₄H₂₆N₂O₃ | 390.48 |
| 5986994 | 11124 | H4 | C₂₆H₂₁ClN₂O₂ | 428.91 |
| 5990041 | 10866 | H7 | C₂₅H₂₅BrN₂O₂•C₂H₂O₄ | 555.42 |
| 5990137 | 11127 | D4 | C₂₆H₂₈N₂O₃ | 416.51 |
| 5993048 | 11127 | G5 | C₁₅H₉BrN₂O₃S | 377.21 |
| 5998734 | 11235 | F11 | C₂₅H₂₆N₂O₂ | 386.49 |
| 6000398 | 11129 | H6 | C₂₅H₁₈N₄O₃ | 422.44 |
| 6015090 | 11248 | G2 | C₂₃H₁₇ClN₂O₄ | 420.85 |
| 6033352 | 10866 | H8 | C₂₈H₃₁N₃O₃•C₂H₂O₄ | 547.6 |
| 6034397 | 10747 | H11 | C₂₇H₂₉ClN₂O₄ | 490.98 |
| 6034674 | 11087 | B8 | C₂₆H₂₆N₄O₃ | 442.51 |
| 6035098 | 11146 | E2 | C₂₆H₂₈N₂O₃ | 416.51 |
| 6035728 | 11032 | D10 | C₂₄H₂₃N₃O₄ | 417.46 |
| 6037360 | 10931 | C2 | C₂₄H₂₃ClN₂O | 390.91 |
| 6038391 | 11144 | H3 | C₂₅H₂₄N₂O₃ | 400.47 |
| 6043815 | 11171 | H10 | C₂₈H₃₁N₃O₄ | 473.57 |
| 6044350 | 11150 | A4 | C₃₃H₃₀N₂O₃ | 502.61 |
| 6044525 | 11150 | H6 | C₂₆H₂₂ClN₃O₄ | 475.93 |
| 6044626 | 11150 | A2 | C₂₈H₃₃NO₆ | 479.57 |
| 6044673 | 11150 | G2 | C₂₈H₂₈N₂O₃ | 440.54 |
| 6044860 | 11149 | H10 | C₂₇H₂₅BrN₂O₃ | 505.4 |
| 6045012 | 11149 | A3 | C₂₂H₂₁NO₄ | 363.41 |
| 6046070 | 11152 | A10 | C₂₇H₂₆N₂O₂ | 410.51 |
| 6046818 | 11152 | H3 | C₂₆H₂₃ClN₂O₂ | 430.93 |
| 6048306 | 11156 | D8 | C₂₈H₂₇BrN₂O₄ | 535.43 |
| 6048935 | 11160 | A7 | C₂₇H₂₆N₂O₂ | 410.51 |
| 6049010 | 11158 | A4 | C₂₅H₂₇NO₄ | 405.49 |
| 6049184 | 11157 | F10 | C₂₅H₂₂Cl₂N₂O₃ | 469.36 |
| 6049448 | 11156 | D10 | C₂₈H₂₇ClN₂O₄ | 490.98 |
| 6056592 | 11165 | A3 | C₂₄H₁₉N₃O | 365.43 |
| 6060848 | 11166 | G8 | C₂₁H₁₇N₃OS | 359.45 |
| 6062505 | 11169 | A7 | C₂₆H₂₂N₂O | 378.47 |
| 6065757 | 11179 | D6 | C₂₈H₂₇N₃O₂ | 437.57 |
| 6066936 | 11173 | H6 | C₁₁H₈ClF₂NO₄ | 291.63 |
| 6068189 | 11176 | G3 | C₂₄H₂₁BrN₂O₂S | 481.41 |
| 6068602 | 11175 | H7 | C₂₂H₂₂N₂O | 330.42 |
| 6069474 | 10654 | H5 | C₂₆H₂₄N₂O | 380.48 |
| 6070379 | 10654 | D6 | C₃₃H₃₃NO₆ | 539.62 |
| 6073875 | 11179 | C2 | C₂₅H₂₁BrN₂O | 445.35 |
| 6074259 | 11181 | G2 | C₂₇H₂₁F₃N₂O₃ | 478.46 |
| 6074532 | 11181 | H3 | C₂₈H₂₈N₂O₃S | 472.6 |
| 6074891 | 11182 | G3 | C₂₅H₂₈N₂O₄S | 452.57 |
| 6081028 | 11185 | C7 | C₃₁H₂₅N₃O₂ | 471.55 |
| 6084652 | 11186 | G3 | C₃₂H₂₆N₂O₃ | 486.56 |
| 6094957 | 11195 | H6 | C₂₇H₂₉N₃O₃ | 443.54 |
| 6095577 | 11195 | C3 | C₂₅H₁₈Cl₂N₂O₃ | 465.33 |
| 6095970 | 11190 | E4 | C₂₂H₁₆N₂O₄S₃ | 468.57 |
| 6103983 | 11198 | H3 | C₂₁H₂₁ClN₂O₂S | 400.92 |
| 6104939 | 11196 | A7 | C₂₆H₂₄N₂O₃ | 412.48 |
| 6141576 | 10820 | A4 | C₃₀H₂₇N₃O₄ | 493.56 |
| 6237735 | 11220 | F9 | C₂₈H₂₅F₃N₂O₃ | 494.51 |
| 6237877 | 11220 | C10 | C₃₀H₃₂N₂O₄ | 484.59 |
| 6237973 | 11223 | B5 | C₂₅H₂₀ClFN₂O | 418.89 |
| 6237992 | 11223 | D5 | C₂₉H₃₀N₂O₃ | 454.56 |
| 6238190 | 11220 | H7 | C₃₅H₃₄N₂O₅ | 562.66 |
| 6238246 | 11220 | A8 | C₃₀H₃₂N₂O₄ | 484.59 |
| 6238475 | 11221 | C2 | C₂₈H₂₅F₃N₂O₃ | 494.51 |
| 6238767 | 11221 | C8 | C₂₈H₂₈N₂O₂ | 424.54 |
| 6239048 | 11221 | H7 | C₂₅H₂₀ClN₃O₃ | 445.9 |
| 6239252 | 11221 | H9 | C₂₆H₂₁N₃O₄ | 439.46 |
| 6239507 | 11221 | B11 | C₃₃H₃₀N₂O₄ | 518.61 |
| 6239538 | 11221 | H11 | C₂₇H₂₅BrN₂O₃ | 505.4 |
| 6239939 | 11223 | G4 | C₂₇H₂₄N₂O₃ | 424.49 |
| 6241376 | 11224 | G4 | C₂₇H₂₆N₂O₂ | 410.51 |
| 6368931 | 11224 | D8 | C₂₅H₂₀BrFN₂O | 463.34 |
| 6370710 | 10901 | C8 | C₃₁H₃₄N₂O₄ | 498.62 |

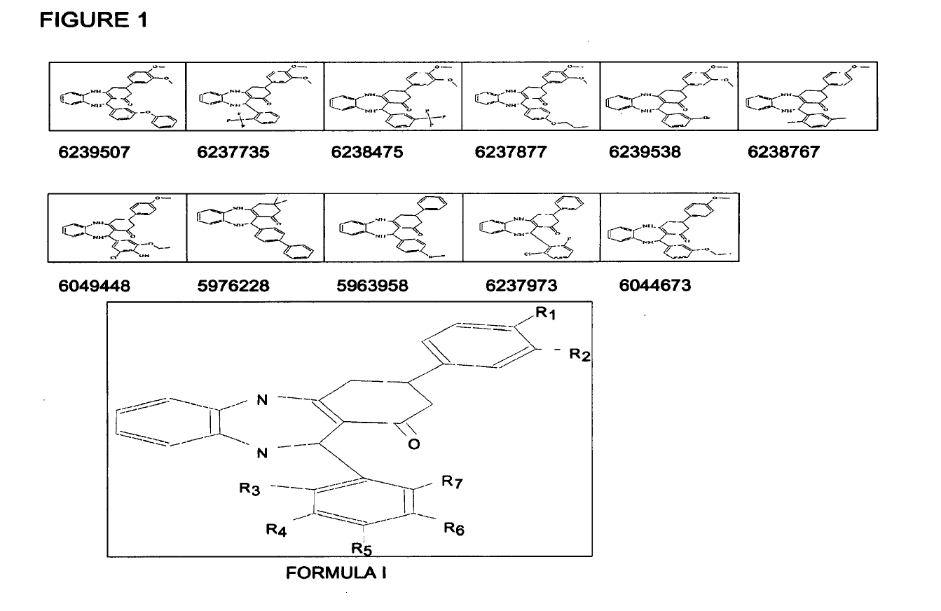

A number of the hit compounds fall into groups with related structures. ER stress inhibitory compounds include but are not limited to the compounds of Formula I (shown in FIG. 1), wherein:

R1 and R2 is each independently selected from the group consisting of hydrogen, alkyl, aryl, heteroaryl, alkoxy, aryloxy, and heteroaryloxy;

R2 is selected from the group consisting of hydrogen, alkyl, aryl, heteroaryl, alkoxy, aryloxy, and heteroaryloxy;

R3-R7 is each independently selected from the group consisting of hydrogen, alkyl, aryl, heteroaryl, alkoxy, aryloxy, heteroaryloxy, halo, and haloalkyl.

Formula I includes without limitation the benzodiazepinone compounds listed in Table 2 below (also referred to herein as Group 1 compounds).

The compounds of Group I (listed according to their ChemBridge compound ID numbers) and their potency data IC₅₀ (μM) are provided in Table 2 below Substituent groups R1-R7 for the compounds of Formula I are also provided in Table 2.

TABLE 2
Potency data for analogs in the benzodiazepinone series
of ER stress-active compounds (Group I compounds).

| Com- | | | | | | | | |
|---|---|---|---|---|---|---|---|---|
| pound | R1 | R2 | R3 | R4 | R5 | R6 | R7 | IC₅₀ (μM)* |
| 6239507 | OMe | OMe | | | | OPh | | 13.89 ± 0.2596, |
| | | | | | | | | 10.17 ± 1.621 |
| 6237735 | OMe | OMe | CF₃ | | | | | 17.16 ± 0.4705, |
| | | | | | | | | 12.5 ± 0.1859 |
| 6238475 | OMe | OMe | | | | CF₃ | | 18.81 ± 0.1566, |
| | | | | | | | | 12.5 ± 0.1695 |
| 6237877 | OMe | OMe | | | O—n-Pr | | | 16.26 ± 0.4393, |
| | | | | | | | | 12.5 ± 0.1695 |
| 6239538 | OMe | OMe | | | | Br | | 15.48 ± 0.2427, |
| | | | | | | | | 12.5 ± 0.1766 |
| 6238767 | OMe | | Me | | | Me | | 13.31 ± 1.1719, |
| | | | | | | | | 12.5 ± 0.1769 |
| 6049448 | OMe | | | Cl | OH | OEt | | 24.83 ± 2.031, |
| | | | | | | | | 23.73 ± 0.642 |
| 5963958 | | | | | SMe | | | 18.55 ± 9.14, |
| | | | | | | | | 12.5 ± 0.1789 |
| 6237973 | | | Cl | | | | F | 22.58 ± 0.6804, |
| | | | | | | | | 19.49 ± 0.1691 |
| 6044673 | OMe | | | | | OEt | | 20.15 ± 0.7806, |
| | | | | | | | | 21.15 ± 0.3619 |

*The IC₅₀ value in bold is from a second assay.

ER stress inhibitory compounds also include but are not limited to the compounds that are structurally similar to the Group 1 compounds, including but not limited to the compounds listed in Table 3 below.

TABLE 3
Compounds sharing structural similarity with Group 1 compounds

| ChemBridge | |
|---|---|
| ID Number | Name |
| 5957532 | [1]11-(4-methoxyphenyl)-3-phenyl-2,3,4,5,10,11-hexahydro-1H- |
| | dibenzo[b,e][1,4]diazepin-1-one |
| 5960775 | [1]11-(3-methoxy-4-propoxyphenyl)-3-phenyl-2,3,4,5,10,11-hexahydro-1H- |
| | dibenzo[b,e][1,4]diazepin-1-one |
| 5963180 | [1]3-(4-methoxyphenyl)-11-(2,3,4-trimethoxyphenyl)-2,3,4,5,10,11-hexahydro-1H- |
| | dibenzo[b,e][1,4]diazepin-1-one |
| 5968938 | [1]11-(4-ethylphenyl)-3-(4-methoxyphenyl)-2,3,4,5,10,11-hexahydro-1H- |
| | dibenzo[b,e][1,4]diazepin-1-one |
| 5969327 | [1]11-(3-chlorophenyl)-3-(4-methoxyphenyl)-2,3,4,5,10,11-hexahydro-1H- |
| | dibenzo[b,e][1,4]diazepin-1-one |
| 5971890 | [1]3-(4-methoxyphenyl)-11-(3-pyridinyl)-2,3,4,5,10,11-hexahydro-1H- |
| | dibenzo[b,e][1,4]diazepin-1-one |
| 5975268 | [1]11-(3-bromophenyl)3-(4-methoxyphenyl)-2,3,4,5,10,11-hexahydro-1H- |
| | dibenzo[b,e][1,4]diazepin-1-one |
| 5977444 | [1]11-(5-bromo-2-methoxyphenyl)-3-(4-chlorophenyl)-2,3,4,5,10,11-hexahydro-1H- |
| | dibenzo[b,e][1,4]diazepin-1-one |
| 5979817 | [1]11-(3-fluorophenyl)-3-(4-methoxyphenyl)-2,3,4,5,10,11-hexahydro-1H- |
| | dibenzo[b,e][1,4]diazepin-1-one |
| 5983297 | [1]3-(4-chlorophenyl)-11-(2,3,4-trimethoxyphenyl)-2,3,4,5,10,11-hexahydro-1H- |
| | dibenzo[b,e][1,4]diazepin-1-one |
| 5987806 | [1]3-(4-chlorophenyl)-11-(3,4,5-trimethoxyphenyl)-2,3,4,5,10,11-hexahydro-1H- |
| | dibenzo[b,e][1,4]diazepin-1-one |
| 6043556 | [1]11-(2-isopropoxyphenyl)-3-(4-methoxyphenyl)-2,3,4,5,10,11-hexahydro-1H- |
| | dibenzo[b,e][1,4]diazepin-1-one |
| 6044250 | [1]3-(4-chlorophenyl)-11-(2-methoxyphenyl)-2,3,4,5,10,11-hexahydro-1H- |
| | dibenzo[b,e][1,4]diazepin-1-one |
| 6044269 | [1]11-(2-methoxyphenyl)-3-(4-methoxyphenyl)-2,3,4,5,10,11-hexahydro-1H- |
| | dibenzo[b,e][1,4]diazepin-1-one |
| 6044485 | [1]11-(3,4-dimethoxyphenyl)-3-(4-methoxyphenyl)-2,3,4,5,10,11-hexahydro-1H- |
| | dibenzo[b,e][1,4]diazepin-1-one |
| 6045602 | [1]11-(5-bromo-2-methoxyphenyl)-3-(4-methoxyphenyl)-2,3,4,5,10,11-hexahydro-1H- |
| | dibenzo[b,e][1,4]diazepin-1-one |
| 6045744 | [1]11-(4-hydroxyphenyl)-3-(4-methoxyphenyl)-2,3,4,5,10,11-hexahydro-1H- |
| | dibenzo[b,e][1,4]diazepin-1-one |
| 6046293 | [1]3-(4-methoxyphenyl)-11-(4-propoxyphenyl)-2,3,4,5,10,11-hexahydro-1H- |
| | dibenzo[b,e][1,4]diazepin-1-one |
| 6046301 | [1]3-(4-methoxyphenyl)-11-(4-methylphenyl)-2,3,4,5,10,11-hexahydro-1H- |
| | dibenzo[b,e][1,4]diazepin-1-one |
| 6046330 | [1]11-(4-ethoxy-3-methoxyphenyl)-3-(4-methoxyphenyl)-2,3,4,5,10,11-hexahydro-1H- |
| | dibenzo[b,e][1,4]diazepin-1-one |
| 6046357 | [1]11-(3-ethoxy-4-hydroxyphenyl)-3-(4-methoxyphenyl)-2,3,4,5,10,11-hexahydro-1H- |
| | dibenzo[b,e][1,4]diazepin-1-one |
| 6046475 | [1]11-[4-(benzyloxy)-3-methoxyphenyl]-3-(4-methoxyphenyl)-2,3,4,5,10,11-hexahydro-1H- |
| | dibenzo[b,e][1,4]diazepin-1-one |
| 6046502 | [1]11-(2-ethoxyphenyl)-3-(4-methoxyphenyl)-2,3,4,5,10,11-hexahydro-1H- |
| | dibenzo[b,e][1,4]diazepin-1-one |
| 6047386 | [1]11-(2,5-dimethoxyphenyl)-3-(4-methoxyphenyl)-2,3,4,5,10,11-hexahydro-1H- |
| | dibenzo[b,e][1,4]diazepin-1-one |
| 6047410 | [1]11-(2,3-dimethoxyphenyl)-3-(4-methoxyphenyl)-2,3,4,5,10,11-hexahydro-1H- |
| | dibenzo[b,e][1,4]diazepin-1-one |
| 6048247 | [1]11-(4-ethoxyphenyl)-3-(4-methoxyphenyl)-2,3,4,5,10,11-hexahydro-1H- |
| | dibenzo[b,e][1,4]diazepin-1-one |
| 6048897 | [1]3-(4-methoxyphenyl)-11-(3-methoxy-4-propoxyphenyl)-2,3,4,5,10,11- |
| | hexahydro-1H-dibenzo[b,e][1,4]diazepin-1-one |
| 6048968 | [1]2-methoxy-4-[3-(4-methoxyphenyl)-1-oxo-2,3,4,5,10,11-hexahydro-1H- |
| | dibenzo[b,e][1,4]diazepin-11-yl]phenylacetate |
| 6049070 | [1]3-(4-chlorophenyl)11-(4-methoxyphenyl)-2,3,4,5,10,11-hexahydro-1H- |
| | dibenzo[b,e][1,4]diazepin-1-one |
| 6049557 | [1]2-ethoxy-4-[3-(4-methoxyphenyl)-1-oxo-2,3,4,5,10,11-hexahydro-1H- |
| | dibenzo[b,e][1,4]diazepin-11-yl]phenyl acetate |
| 6059072 | [1]3-(4-methoxyphenyl)-11-phenyl-2,3,4,5,10,11-hexahydro-1H- |
| | dibenzo[b,e][1,4]diazepin-1-one |
| 6065759 | [1]7-benzoyl-11-(3,4-dimethoxyphenyl)-2,3,4,5,10,11-hexahydro-1H- |
| | dibenzo[b,e][1,4]diazepin-1-one |
| 6065762 | [1]7-benzoyl-11-(3-ethoxy-4-hydroxyphenyl)-2,3,4,5,10,11-hexahydro-1H- |
| | dibenzo[b,e][1,4]diazepin-1-one |
| 6067967 | [1]3-(4-methoxyphenyl)-11-(2,4,5-trimethoxyphenyl)-2,3,4,5,10,11-hexahydro-1H- |
| | dibenzo[b,e][1,4]diazepin-1-one |
| 6070404 | [1]3-(4-chlorophenyl)-11-(2,5-dimethoxyphenyl)-2,3,4,5,10,11-hexahydro-1H- |
| | dibenzo[b,e][1,4]diazepin-1-one |
| 6070528 | [1]11-(2-bromo-4,5-dimethoxyphenyl)-3-(4-methoxyphenyl)-2,3,4,5,10,11-hexahydro-1H- |
| | dibenzo[b,e][1,4]diazepin-1-one |
| 6072098 | [1]3-(3,4-dimethoxyphenyl)-11-(4-fluorophenyl)-2,3,4,5,10,11-hexahydro-1H- |
| | dibenzo[b,e][1,4]diazepin-1-one |
| 6072311 | [1]3-(3,4-dimethoxyphenyl)-11-(2-ethoxyphenyl)-2,3,4,5,10,11-hexahydro-1H- |
| | dibenzo[b,e][1,4]diazepin-1-one |
| 6076307 | [1]11-(2-chlorophenyl)-3-(3,4-dimethoxyphenyl)-2,3,4,5,10,11-hexahydro-1H- |
| | dibenzo[b,e][1,4]diazepin-1-one |
| 6076444 | [1]11-(4-chlorophenyl)-3-(3,4-dimethoxyphenyl)-2,3,4,5,10,11-hexahydro-1H- |
| | dibenzo[b,e][1,4]diazepin-1-one |
| 6078444 | [1]3-(3,4-dimethoxyphenyl)-11-(4-methoxyphenyl)-2,3,4,5,10,11-hexahydro-1H- |
| | dibenzo[b,e][1,4]diazepin-1-one |
| 6078781 | [1]11-(3,4-dimethoxyphenyl)-3-phenyl-2,3,4,5,10,11-hexahydro-1H- |
| | dibenzo[b,e][1,4]diazepin-1-one |
| 6079090 | [1]11-(3,4-difluorophenyl)-3-(4-methoxyphenyl)-2,3,4,5,10,11-hexahydro-1H- |
| | dibenzo[b,e][1,4]diazepin-1-one |
| 6079475 | [1]3-(4-chlorophenyl)-11-(2,3-dimethoxyphenyl)-2,3,4,5,10,11-hexahydro-1H- |
| | dibenzo[b,e][1,4]diazepin-1-one |
| 6079686 | [1]3-(4-chlorophenyl)-11-(3,4-dimethoxyphenyl)-2,3,4,5,10,11-hexahydro-1H- |
| | dibenzo[b,e][1,4]diazepin-1-one |
| 6079798 | [1]3-(3,4-dimethoxyphenyl)-11-(2-fluorophenyl)-2,3,4,5,10,11-hexahydro-1H- |
| | dibenzo[b,e][1,4]diazepin-1-one |
| 6081438 | [1]7-benzoyl-11-(4-hydroxy-3-methoxyphenyl)-2,3,4,5,10,11-hexahydro-1H- |
| | dibenzo[b,e][1,4]diazepin-1-one |
| 6082830 | [1]7-benzoyl-11-(4-methoxyphenyl)-3-phenyl-2,3,4,5,10,11-hexahydro-1H- |
| | dibenzo[b,e][1,4]diazepin-1-one |
| 6083310 | [1]11-(4-hydroxy-3-methoxyphenyl)-8-methyl-3-phenyl-2,3,4,5,10,11-hexahydro-1H- |
| | dibenzo[b,e][1,4]diazepin-1-one |
| 6085053 | [1]11-(4-hydroxyphenyl)-8-methyl-3-phenyl-2,3,4,5,10,11-hexahydro-1H- |
| | dibenzo[b,e][1,4]diazepin-1-one |
| 6085274 | [1]7-benzoyl-11-(4-hydroxy-3-methoxyphenyl)-3-phenyl-2,3,4,5,10,11-hexahydro-1H- |
| | dibenzo[b,e][1,4]diazepin-1-one |
| 6105705 | [1]3-(3,4-dimethoxyphenyl)-11-(4-methylphenyl)-2,3,4,5,10,11-hexahydro-1H- |
| | dibenzo[b,e][1,4]diazepin-1-one |
| 6126209 | [1]3-(2-ethoxyphenyl)-11-(4-methoxyphenyl)-2,3,4,5,10,11-hexahydro-1H- |
| | dibenzo[b,e][1,4]diazepin-1-one |
| 6130287 | [1]11-(4-methoxyphenyl)-3-(2-propoxyphenyl)-2,3,4,5,10,11-hexahydro-1H- |
| | dibenzo[b,e][1,4]diazepin-1-one |
| 6237639 | [1]3-(3,4-dimethoxyphenyl)-11-(3-pyridinyl)-2,3,4,5,10,11-hexahydro-1H- |
| | dibenzo[b,e][1,4]diazepin-1-one |
| 6237685 | [1]11-(3-chloro-4-hydroxy-5-methoxyphenyl)-3-phenyl-2,3,4,5,10,11-hexahydro-1H- |
| | dibenzo[b,e][1,4]diazepin-1-one |
| 6238189 | [1]11-(2-methoxyphenyl)-3-phenyl-2,3,4,5,10,11-hexahydro-1H- |
| | dibenzo[b,e][1,4]diazepin-1-one |
| 6238271 | [1]11-(2,3-dimethoxyphenyl)-3-(3,4-dimethoxyphenyl)-2,3,4,5,10,11-hexahydro-1H- |
| | dibenzo[b,e][1,4]diazepin-1-one |
| 6238304 | [1]11-(3,4-difluorophenyl)-3-(3,4-dimethoxyphenyl)-2,3,4,5,10,11-hexahydro-1H- |
| | dibenzo[b,e][1,4]diazepin-1-one |
| 6238473 | [1]7-benzoyl-11-(3,4,5-trimethoxyphenyl)-2,3,4,5,10,11-hexahydro-1H- |
| | dibenzo[b,e][1,4]diazepin-1-one |
| 6238508 | [1]4-[3-(3,4-dimethoxyphenyl)-1-oxo-2,3,4,5,10,11-hexahydro-1H- |
| | dibenzo[b,e][1,4]diazepin-11-yl]-2-methoxyphenyl acetate |
| 6238763 | [1]11-(5-bromo-2-methoxyphenyl)-3-(3,4-dimethoxyphenyl)-2,3,4,5,10,11-hexahydro-1H- |
| | dibenzo[b,e][1,4]diazepin-1-one |
| 6239093 | [1]11-(3-bromo-4,5-dimethoxyphenyl)-3-(4-methoxyphenyl)-2,3,4,5,10,11-hexahydro-1H- |
| | dibenzo[b,e][1,4]diazepin-1-one |
| 6239790 | [1]11-(3-bromo-4-fluorophenyl)-3-(4-methoxyphenyl)-2,3,4,5,10,11-hexahydro-1H- |
| | dibenzo[b,e][1,4]diazepin-1-one |
| 6239945 | [1]11-(4-biphenylyl)-3-(3,4-dimethoxyphenyl)-2,3,4,5,10,11-hexahydro-1H- |
| | dibenzo[b,e][1,4]diazepin-1-one |
| 6240138 | [1]3-(3,4-dimethoxyphenyl)-11-(1-naphthyl)-2,3,4,5,10,11-hexahydro-1H- |
| | dibenzo[b,e][1,4]diazepin-1-one |
| 6240144 | [1]11-(2-bromophenyl)-3-(3,4-dimethoxyphenyl)-2,3,4,5,10,11-hexahydro-1H- |
| | dibenzo[b,e][1,4]diazepin-1-one |
| 6240442 | [1]11-(2,3-dichlorophenyl)-3-(3,4-dimethoxyphenyl)-2,3,4,5,10,11-hexahydro-1H- |
| | dibenzo[b,e][1,4]diazepin-1-one |
| 6241188 | [1]3-(3,4-dimethoxyphenyl)-11-(2-isopropoxyphenyl)-2,3,4,5,10,11-hexahydro-1H- |
| | dibenzo[b,e][1,4]diazepin-1-one |
| 6241409 | [1]11-[2-(benzyloxy)phenyl]-3-(3,4-dimethoxyphenyl)-2,3,4,5,10,11-hexahydro-1H- |
| | dibenzo[b,e][1,4]diazepin-1-one |
| 6293385 | [1]11-(4-hydroxy-3-methoxyphenyl)-2,3,4,5,10,11-hexahydro-1H- |
| | dibenzo[b,e][1,4]diazepin-1-one |
| 6367396 | [1]N-{4-[3-(3,4-dimethoxyphenyl)-1-oxo-2,3,4,5,10,11-hexahydro-1H- |
| | dibenzo[b,e][1,4]diazepin-11-yl]phenyl}acetamide |
| 6369106 | [1]11-(2,5-difluorophenyl)-3-(3,4-dimethoxyphenyl)-2,3,4,5,10,11-hexahydro-1H- |
| | dibenzo[b,e][1,4]diazepin-1-one |
| 6369387 | [1]11-(3,4-dichlorophenyl)-3-(3,4-dimethoxyphenyl)-2,3,4,5,10,11-hexahydro-1H- |
| | dibenzo[b,e][1,4]diazepin-1-one |
| 6369617 | [1]11-(4-hydroxy-3,5-dimethoxyphenyl)-3-phenyl-2,3,4,5,10,11-hexahydro-1H- |
| | dibenzo[b,e][1,4]diazepin-1-one |
| 6369841 | [1]3-(3,4-dimethoxyphenyl)-11-(2-methylphenyl)-2,3,4,5,10,11-hexahydro-1H- |
| | dibenzo[b,e][1,4]diazepin-1-one |
| 6370064 | [1]11-(2,4-dimethoxyphenyl)-3-phenyl-2,3,4,5,10,11-hexahydro-1H- |
| | dibenzo[b,e][1,4]diazepin-1-one |
| 6370403 | [1]11-(3-bromo-4-methoxyphenyl)-3-phenyl-2,3,4,5,10,11-hexahydro-1H- |
| | dibenzo[b,e][1,4]diazepin-1-one |
| 6370459 | [1]3-(3,4-dimethoxyphenyl)-11-(4-hydroxyphenyl)-2,3,4,5,10,11-hexahydro-1H- |
| | dibenzo[b,e][1,4]diazepin-1-one |
| 6370590 | [1]3-(2-chlorophenyl)-11-(2,3-dimethoxyphenyl)-2,3,4,5,10,11-hexahydro-1H- |
| | dibenzo[b,e][1,4]diazepin-1-one |
| 6370595 | [1]3-(3,4-dimethoxyphenyl)-11-(3-fluorophenyl)-2,3,4,5,10,11-hexahydro-1H- |
| | dibenzo[b,e][1,4]diazepin-1-one |
| 6371110 | [1]3-phenyl-11-(2,4,5-trimethoxyphenyl)-2,3,4,5,10,11-hexahydro-1H- |
| | dibenzo[b,e][1,4]diazepin-1-one |
| 6404032 | [1]11-(4-hydroxy-3,5-dimethoxyphenyl)-2,3,4,5,10,11-hexahydro-1H- |
| | dibenzo[b,e][1,4]diazepin-1-one |
| 6424496 | [1]11-(3,4-dimethoxyphenyl)-2,3,4,5,10,11-hexahydro-1H- |
| | dibenzo[b,e][1,4]diazepin-1-one |
| 6430614 | [1]3-(2-chlorophenyl)-11-(2,4-dimethoxyphenyl)-2,3,4,5,10,11-hexahydro-1H- |
| | dibenzo[b,e][1,4]diazepin-1-one |
| 6430633 | [1]3-(2-chlorophenyl)-11-(2,4,5-trimethoxyphenyl)-2,3,4,5,10,11-hexahydro-1H- |
| | dibenzo[b,e][1,4]diazepin-1-one |
| 6434915 | [1]3-(2-chlorophenyl)-11-(3-hydroxy-4-methoxyphenyl)-2,3,4,5,10,11-hexahydro-1H- |
| | dibenzo[b,e][1,4]diazepin-1-one |
| 6435244 | [1]3-(2-chlorophenyl)-11-(2,5-dimethoxyphenyl)-2,3,4,5,10,11-hexahydro-1H- |
| | dibenzo[b,e][1,4]diazepin-1-one |
| 7098684 | [1]7-benzoyl-11-(3-hydroxy-4-methoxyphenyl)-2,3,4,5,10,11-hexahydro-1H- |
| | dibenzo[b,e][1,4]diazepin-1-one |
| 7117433 | [1]3-(2-chlorophenyl)-11-(4-hydroxy-3-methoxyphenyl)-2,3,4,5,10,11-hexahydro-1H- |
| | dibenzo[b,e][1,4]diazepin-1-one |
| 7353449 | [1]3-(4-ethoxy-3-methoxyphenyl)-11-(3-hydroxy-4-methoxyphenyl)-2,3,4,5,10,11-hexahydro-1H- |
| | dibenzo[b,e][1,4]diazepin-1-one |
| 7353985 | [1]11-(2,3-dimethoxyphenyl)-3-(4-methylphenyl)-2,3,4,5,10,11-hexahydro-1H- |
| | dibenzo[b,e][1,4]diazepin-1-one |
| 7355097 | [1]11-(4-chlorophenyl)-3-(3,4,5-trimethoxyphenyl)-2,3,4,5,10,11-hexahydro-1H- |
| | dibenzo[b,e][1,4]diazepin-1-one |
| 7358383 | [1]11-(3-fluorophenyl)-3-(3-methoxyphenyl)-2,3,4,5,10,11-hexahydro-1H- |
| | dibenzo[b,e][1,4]diazepin-1-one |
| 7359723 | [1]11-(2-fluorophenyl)-3-(3,4,5-trimethoxyphenyl)-2,3,4,5,10,11-hexahydro-1H- |
| | dibenzo[b,e][1,4]diazepin-1-one |
| 7359797 | [1]methyl4-[3-(3-methoxyphenyl)-1-oxo-2,3,4,5,10,11-hexahydro-1H- |
| | dibenzo[b,e][1,4]diazepin-11-yl]benzoate |
| 7361320 | [1]11-(4-pyridinyl)-3-(3,4,5-trimethoxyphenyl)-2,3,4,5,10,11-hexahydro-1H- |
| | dibenzo[b,e][1,4]diazepin-1-one |
| 7391086 | [1]11-(2-hydroxy-3-methoxyphenyl)-3-(4-methylphenyl)-2,3,4,5,10,11-hexahydro-1H- |
| | dibenzo[b,e][1,4]diazepin-1-one |
| 7464599 | [1]11-(4-hydroxy-3,5-dimethoxyphenyl)-3-(4-methylphenyl)-2,3,4,5,10,11-hexahydro-1H- |
| | dibenzo[b,e][1,4]diazepin-1-one |
| 7967255 | [1]11-(2,4-dimethoxyphenyl)-3-(4-fluorophenyl)-2,3,4,5,10,11-hexahydro-1H- |
| | dibenzo[b,e][1,4]diazepin-1-one |
| 7970252 | [1]11-(2,5-difluorophenyl)-3-(2,5-dimethoxyphenyl)-2,3,4,5,10,11-hexahydro-1H- |
| | dibenzo[b,e][1,4]diazepin-1-one |
| 9104830 | [1]3-(3-methoxyphenyl)-11-(3-pyridinyl)-2,3,4,5,10,11-hexahydro-1H- |
| | dibenzo[b,e][1,4]diazepin-1-one |

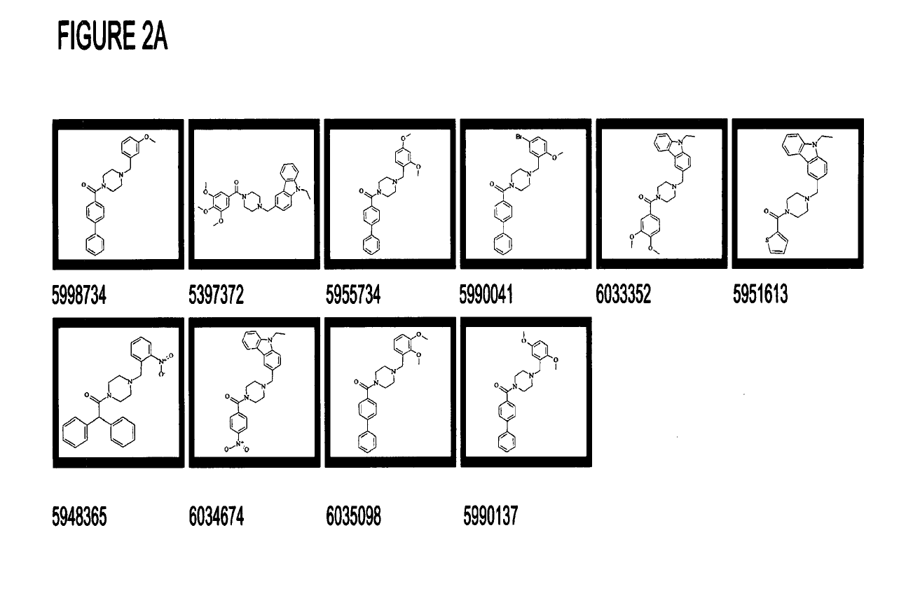

ER stress inhibitory compounds include but are not limited to the compounds of Formula II-1 (Group 2-1 compounds) and Formula II-2 (Group 2-2 compounds) below, as shown in FIG. 2B. (Group 2-1 compounds and Group 2-2 compounds are collectively referred to as Group 2 compounds herein.)

For Formula II-1, R1-R7 is each independently selected from the group consisting of hydrogen, alkyl, aryl, heteroaryl, alkoxy, aryloxy, heteroaryloxy, halo, and haloalkyl.

For Formula II-2, R is selected from the group consisting of hydrogen, alkyl, aryl, heteroaryl, alkoxy, aryloxy, heteroaryloxy, halo, and haloalkyl.

Representative compounds of Group 2-1 and Group 2-2 (listed according to their ChemBridge compound ID numbers) and their potency data [IC₅₀ (μM)] are provided in Tables 4 and 5 below. For these selected compounds of Formula II-1 (Group 2-1), substituent groups R1-R7 are provided for each compound in Table 4. For these selected compounds of Formula II-2 (Group 2-2), substituent group R is provided for each compound in Table 5.

TABLE 4
Potency data for ER stress-active compounds of Group 2-1.

| Com- | | | | | | | | |
|---|---|---|---|---|---|---|---|---|
| pound | R1 | R2 | R3 | R4 | R5 | R6 | R7 | IC₅₀ (μM)* |
| 5998734 | | | OMe | | | Ph | | 20.02 ± 0.4928, |
| | | | | | | | | 14.63 ± 26.56 |
| 5955734 | | OMe | | OMe | | Ph | | 23.8, |
| | | | | | | | | 16.53 ± 1.467 |
| 5990041 | Br | | | OMe | | Ph | | 12.85 ± 0.3183, |
| | | | | | | | | 14.25 ± 1.204 |
| 6035098 | | | OMe | OMe | | Ph | | 19.75 ± 0.7206, |
| | | | | | | | | 16.63 ± 2.032 |
| 5990137 | OMe | | | OMe | | Ph | | 17.52 ± 0.6747, |
| | | | | | | | | 13.86 ± 1.059 |

*The IC₅₀ value in bold is from a second assay.

TABLE 5
Potency data for ER stress-active compounds of Group 2-2.

| Compound | R | IC₅₀ (μM)* |
|---|---|---|
| 5397372 | 3,4,5-methoxyphenyl | 18.89 ± 0.5683, |
| | | 13.47 ± 2.024 |
| 6033352 | 3,4-methoxy | 13.43 ± 0.2476, |
| | | 13.77 ± 0.5476 |
| 6034674 | p-nitrophenyl | 15.01 ± 0.4953, |
| | | 11.53 ± 0.5013 |
| 5951613 | 2-thiophene | 16.74 ± 0.1577, |
| | | 12.72 ± 1.106 |

*The IC₅₀ value in bold is from a second assay.

Examples of compounds that are structurally similar with the Group 2 compounds are provided in Table 6.

TABLE 6
Compounds sharing structural similarity with Group 2 compounds

| ChemBridge | |
|---|---|
| ID Number | Name |
| 5254057 | [1]1-(4-biphenylylcarbonyl)-4-methylpiperazine |
| 5348446 | [1]1-benzyl-4-(3-methylbenzoyl)piperazine |
| 5525833 | [1]1-benzyl-4-(4-ethoxybenzoyl)piperazine |
| 5539718 | [1]1-benzyl-4-(3-methoxybenzoyl)piperazine |
| 5541280 | [1]1-(diphenylmethyl)-4-(3-methoxybenzoyl)piperazine |
| 5654134 | [1]1-(4-biphenylylcarbonyl)-4-phenylpiperazine |
| 5663730 | [1]1-benzyl-4-(4-biphenylylcarbonyl)piperazine |
| 5817217 | [1]1-benzoyl-4-(3,4-dimethoxybenzyl)piperazine |
| 5817458 | [1]1-(3-bromobenzyl)-4-(3-methoxybenzoyl)piperazine |
| 5818397 | [1]1-(4-methoxybenzoyl)-4-(3-methoxybenzyl)piperazine |
| 5818425 | [1]1-(4-fluorobenzoyl)-4-(3-methoxybenzyl)piperazine |
| 5819704 | [1]1-(3-methoxybenzoyl)-4-(4-methylbenzyl)piperazine |
| 5822650 | [1]1-benzoyl-4-(4-methoxybenzyl)piperazine |
| 5823862 | [1]1-(3-bromobenzyl)-4-(4-methoxybenzoyl)piperazine |
| 5824302 | [1]1-(3-methoxybenzoyl)-4-(3-methoxybenzyl)piperazine |
| 5826486 | [1]1-(4-methoxybenzoyl)-4-(4-methylbenzyl)piperazine |
| 5827101 | [1]1-(3-methoxybenzoyl)-4-(2-methylbenzyl)piperazine |
| 5828983 | [1]1-(3-methoxybenzoyl)-4-(4-pyridinylmethyl)piperazine |
| 5830113 | [1]1-(3-fluorobenzoyl)-4-(3-methoxybenzyl)piperazine |
| 5830528 | [1]1-(3-methoxybenzoyl)-4-(4-methoxybenzyl)piperazine |
| 5831931 | [1]1-isonicotinoyl-4-(3-methoxybenzyl)piperazine |
| 5832526 | [1]1-benzoyl-4-(3-methoxybenzyl)piperazine |
| 5833328 | [1]1-(3-fluorobenzoyl)-4-(4-methoxybenzyl)piperazine |
| 5833363 | [1]1-(4-bromobenzyl)-4-(3-methoxybenzoyl)piperazine |
| 5941781 | [1]1-(4-biphenylylcarbonyl)-4-(3-methoxybenzoyl)piperazine |
| 5944032 | [1]1-(4-biphenylylcarbonyl)-4-(4-methoxybenzoyl)piperazine |
| 5948821 | [1]1-(3-chlorobenzoyl)-4-(4-methoxybenzyl)piperazine |
| 5948826 | [1]1-(4-biphenylylcarbonyl)-4-(3-pyridinylmethyl)piperazine |
| 5949252 | [1]1-(4-chlorobenzyl)-4-(3-methoxybenzoyl)piperazine |
| 5949497 | [1]1-(4-biphenylylcarbonyl)-4-(3-ethoxy-4-methoxybenzyl)piperazine |
| 5949609 | [1]1-(4-fluorobenzoyl)-4-(3-methylbenzyl)piperazine |
| 5950588 | [1]1-(4-methoxybenzoyl)-4-(3-methylbenzyl)piperazine |
| 5951626 | [1]1-(4-ethylbenzyl)-4-(3-methoxybenzoyl)piperazine |
| 5951693 | [1]1-(4-biphenylylcarbonyl)-4-(3-fluorobenzyl)piperazine |
| 5952657 | [1]1-(4-biphenylylcarbonyl)-4-(4-bromobenzyl)piperazine |
| 5953086 | [1]1-(4-biphenylylcarbonyl)-4-(2-pyridinylmethyl)piperazine |
| 5956659 | [1]1-isonicotinoyl-4-(3-phenoxybenzyl)piperazine |
| 5980954 | [1]1-benzoyl-4-(4-biphenylylcarbonyl)piperazine |
| 5990076 | [1]1-(4-biphenylylcarbonyl)-4-(4-ethoxybenzyl)piperazine |
| 5991082 | [1]1-(3-chlorobenzyl)-4-(4-methoxybenzoyl)piperazine |
| 5991155 | [1]1-(4-fluorobenzyl)-4-(3-methoxybenzoyl)piperazine |
| 5992370 | [1]1-(3-fluorobenzyl)-4-(4-methoxybenzoyl)piperazine |
| 5994081 | [1]1-(4-biphenylylcarbonyl)-4-(2-bromobenzyl)piperazine |
| 5996087 | [1]1-(4-biphenylylcarbonyl)-4-(2-methoxybenzyl)piperazine |
| 5996484 | [1]1-(3-chlorobenzoyl)-4-(3-methoxybenzyl)piperazine |
| 5999571 | [1]1-(4-fluorobenzoyl)-4-(3-fluorobenzyl)piperazine |
| 6033085 | [1]1-(4-fluorobenzoyl)-4-(3-phenoxybenzyl)piperazine |
| 6034736 | [1]1-(4-biphenylylcarbonyl)-4-(3,4-dimethoxybenzyl)piperazine |
| 6037179 | [1]1-benzoyl-4-(3-phenoxybenzyl)piperazine |
| 6038779 | [1]1-(4-biphenylylcarbonyl)-4-(4-pyridinylmethyl)piperazine |
| 6038942 | [1]1-(4-biphenylylcarbonyl)-4-(3-chlorobenzyl)piperazine |
| 6039093 | [1]1-(3-fluorobenzoyl)-4-(3-phenoxybenzyl)piperazine |
| 6039171 | [1]1-(4-biphenylylcarbonyl)-4-(3-bromobenzyl)piperazine |
| 6040003 | [1]1-(4-biphenylylcarbonyl)-4-(4-ethylbenzyl)piperazine |
| 6040469 | [1]1-(3-chlorobenzyl)-4-(3-methoxybenzoyl)piperazine |
| 6040761 | [1]1-(4-ethylbenzyl)-4-(4-methoxybenzoyl)piperazine |
| 6040955 | [1]1-(4-biphenylylcarbonyl)-4-[4-(methylthio)benzyl]piperazine |
| 6813600 | [1]1-(4-biphenylylcarbonyl)-4-ethylpiperazine |
| 7944537 | [1]1-(3-methoxybenzyl)-4-(2-methylbenzoyl)piperazine |
| 7945326 | [1]1-(4-biphenylylcarbonyl)-4-propylpiperazine |
| 7946459 | [1]1-(4-biphenylylcarbonyl)-4-(3-methoxyphenyl)piperazine |

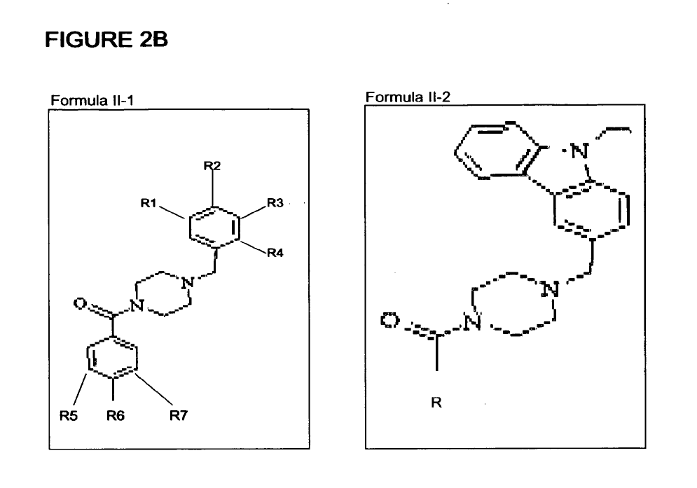

FIG. 3 shows the structures of five independent compounds that do not fall within the compounds of Formula I or Formula II. These compounds are (listed according to their ChemBridge Compound ID numbers):
5980750
5803884
6049184
5979207
6141576
   Examples of compounds that are structurally similar with these compounds are provided in Tables 7 to 11 below.

TABLE 7
Compounds sharing structural similarity with ChemBridge Compound ID number 5980750

| ChemBridge | |
|---|---|
| ID Number | Name |
| 5486291 | [1]6-(1-piperidinylsulfonyl)benzo[cd]indol-2(1H)-one |
| 5914317 | [0]4-[(dimethylamino)sulfonyl]-N-(1-ethyl-2-oxo-1,2-dihydrobenzo[cd]indol-6-yl)benzamide |
| 5914389 | [1]N-(1-ethyl-2-oxo-1,2-dihydrobenzo[cd]indol-6-yl)-4-(4-morpholinylsulfonyl)benzamide |
| 7702550 | [1]2-(2-oxobenzo[cd]indol-1(2H)-yl)-N-[4-(1-piperidinylsulfonyl)phenyl]acetamide |
| 7753682 | [1]6-(1-azepanylsulfonyl)benzo[cd]indol-2(1H)-one |
| 7810614 | [1]N-[4-(1-azepanylsulfonyl)phenyl]-2-(2-oxobenzo[cd]indol-1(2H)-yl)acetamide |
| 7833747 | [1]2-(2-oxobenzo[cd]indol-1(2H)-yl)-N-[4-(1-pyrrolidinylsulfonyl)phenyl]acetamide |
| 7912320 | [1]6-(1-pyrrolidinylsulfonyl)benzo[cd]indol-2(1H)-one |
| 7917663 | [1]1-methyl-6-(1-piperidinylsulfonyl)benzo[cd]indol-2(1H)-one |
| 7917666 | [1]6-(1-azepanylsulfonyl)-1-ethylbenzo[cd]indol-2(1H)-one |

TABLE 8
Compounds sharing structural similarity with ChemBridge Compound ID number 5803884

| ChemBridge | |
|---|---|
| ID Number | Name |
| 5228473 | [1]1-acetyl-2-methyl-N-phenyl-1,2,3,4-tetrahydro-4-quinolinamine |
| 5228474 | [1]2-methyl-N-phenyl-1-propionyl-1,2,3,4-tetrahydro-4-quinolinamine |
| 5228479 | [1]1-benzoyl-2-methyl-N-phenyl-1,2,3,4-tetrahydro-4-quinolinamine |
| 5228657 | [1]1-butyryl-2-methyl-N-phenyl-1,2,3,4-tetrahydro-4-quinolinamine |
| 5302550 | [1]1-(4-methoxybenzoyl)-2-methyl-N-phenyl-1,2,3,4-tetrahydro-4-quinolinamine |
| 5304079 | [1]2-methyl-N-phenyl-1-(2-thienylcarbonyl)-1,2,3,4-tetrahydro-4-quinolinamine |
| 5306187 | [1]1-(3-methoxybenzoyl)-2-methyl-N-phenyl-1,2,3,4-tetrahydro-4-quinolinamine |
| 5307598 | [1]1-(2-furoyl)-2-methyl-N-phenyl-1,2,3,4-tetrahydro-4-quinolinamine |
| 5373183 | [1]1-cinnamoyl-2-methyl-N-phenyl-1,2,3,4-tetrahydro-4-quinolinamine |
| 5373221 | [1]1-[(4-fluorophenoxy)acetyl]-2-methyl-N-phenyl-1,2,3,4-tetrahydro-4-quinolinamine |
| 5466853 | [1]1-[3-(4-methoxyphenyl)acryloyl]-2-methyl-N-phenyl-1,2,3,4-tetrahydro-4-quinolinamine |
| 5477633 | [1]2-methyl-N-phenyl-1-(tetrahydro-2-furanylcarbonyl)-1,2,3,4-tetrahydro-4-quinolinamine |
| 5481700 | [1]N-{1-[3-(4-methoxyphenyl)acryloyl]-2-methyl-1,2,3,4-tetrahydro-4-quinolinyl}-N-phenylacetamide |
| 5701623 | [1]1-[(4-chlorophenoxy)acetyl]-2-methyl-N-phenyl-1,2,3,4-tetrahydro-4-quinolinamine |
| 5804786 | [1]N-[l-(2-furoyl)-2-methyl-1,2,3,4-tetrahydro-4-quinolinyl]-N-phenyl-2-furamide |
| 5804903 | [1]N-(2-methyl-1,2,3,4-tetrahydro-4-quinolinyl)-N-phenyl-2-furamide |
| 5874506 | [1]2-methyl-1-[(2-naphthyloxy)acetyl]-N-phenyl-1,2,3,4-tetrahydro-4-quinolinamine |
| 6101595 | [1]1-[(4-methoxyphenoxy)acetyl]-2-methyl-N-phenyl-1,2,3,4-tetrahydro-4-quinolinamine |
| 6585676 | [1]4-chloro-N-[1-(2-furoyl)-2-methyl-1,2,3,4-tetrahydro-4-quinolinyl]-N-phenylbenzamide |
| 7189063 | [1]1-(2-furoyl)-2,6-dimethyl-4-phenyl-1,2,3,4-tetrahydroquinoline |
| 7223474 | [1]2-methyl-N-phenyl-1-[3-(3,4,5-trimethoxyphenyl)acryloyl]-1,2,3,4-tetrahydro-4-quinolinamine |
| 7358257 | [1]1-isobutyryl-2-methyl-N-phenyl-1,2,3,4-tetrahydro-4-quinolinamine |

TABLE 9
Compounds sharing structural similarity with ChemBridge Compound ID number 6049184

| ChemBridge | |
|---|---|
| ID Number | Name |
| 5169058 | [1]3-[1-(benzylamino)ethylidene]-1-(2,6-dichlorophenyl)-1,3-dihydro-2H-indol-2-one |
| 5169060 | [1]1-(2,6-dichlorophenyl)-3-(ethoxymethylene)-1,3-dihydro-2H-indol-2-one |
| 5169062 | [1]1-(2,6-dichlorophenyl)-3-[1-(dimethylamino)ethylidene]-1,3-dihydro-2H-indol-2-one |
| 6046072 | [1]1-(2,6-dichlorophenyl)-3-[(dimethylamino)methylene]-2-oxo-6-indolinecarbaldehyde |
| 6048089 | [1]3-(aminomethylene)-1-(2,6-dichlorophenyl)-5-nitro-1,3-dihydro-2H-indol-2-one |
| 6049047 | [1]1-(2,6-dichlorophenyl)-3-{[(3-hydroxypropyl)amino]methylene}-1,3-dihydro-2H-indol-2-one |
| 6050455 | [1]1-(2,6-dichlorophenyl)-3-({[1-(hydroxymethyl)propyl]amino}methylene)-1,3-dihydro-2H-indol-2-one |

TABLE 10
Compounds sharing structural similarity with ChemBridge Compound ID number 5979207

| ChemBridge | |
|---|---|
| ID Number | Name |
| 5963981 | 7-[2-(4-biphenylyl)-2-oxoethyl]-3-(2-thienyl)-6,7-dihydro-5H-imidazo[2,1-b][1,3]thiazol-4-ium bromide |
| 5967192 | 7-(2-oxo-2-phenylethyl)-3-phenyl-5,6-dihydroimidazo[2,1-b][1,3]thiazol-7-ium bromide |
| 5974639 | 7-[2-(4-nitrophenyl)-2-oxoethyl]-6-phenyl-7H-imidazo[2,1-b][1,3]thiazol-4-ium bromide |
| 5977751 | 7-(2-oxo-2-phenylethyl)-6-phenyl-7H-imidazo[2,1-b][1,3]thiazol-4-ium bromide |
| 5981975 | 7-[2-(4-bromophenyl)-2-oxoethyl]-3-(2-thienyl)-6,7-dihydro-5H-imidazo[2,1-b][1,3]thiazol-4-ium bromide |

TABLE 11
Compounds sharing structural similarity with ChemBridge Compound ID number 6141576

| ChemBridge | |
|---|---|
| ID Number | Name |
| 5625226 | [1]3-(4-ethylphenyl)-2-(4-methoxyphenyl)-2,3-dihydro-4(1H)-quinazolinone |
| 6134915 | [1]3-(4-methoxyphenyl)-2-(3,4,5-trimethoxyphenyl)-2,3-dihydro-4(1H)-quinazolinone |
| 6136631 | [1]2-(3,4-dimethoxyphenyl)-3-(4-methoxyphenyl)-2,3-dihydro-4(1H)-quinazolinone |
| 6139622 | [1]N-[4-({2-methoxy-5-[3-(4-methoxyphenyl)-4-oxo-1,2,3,4-tetrahydro-2-quinazolinyl]benzyl}oxy)phenyl]acetamide |
| 6148245 | [1]2-{4-methoxy-3-[(2-pyridinyloxy)methyl]phenyl}-3-phenyl-2,3-dihydro-4(1H)-quinazolinone |
| 6149128 | [1]2-(2,4-dimethoxyphenyl)-3-(4-methoxyphenyl)-2,3-dihydro-4(1H)-quinazolinone |
| 6538397 | [1]2-(4-ethylphenyl)-3-(4-methoxyphenyl)-2,3-dihydro-4(1H)-quinazolinone |
| 6888648 | [1]methyl4-[3-(4-methoxyphenyl)-4-oxo-1,2,3,4-tetrahydro-2-quinazolinyl]benzoate |
| 6955545 | [1]N-[4-(4-oxo-3-phenyl-1,2,3,4-tetrahydro-2-quinazolinyl)phenyl]acetamide |
| 7114788 | [1]N-{4-[3-(2-methoxyphenyl)-4-oxo-1,2,3,4-tetrahydro-2-quinazolinyl]phenyl}acetamide |
| 7232061 | [1]2-{3-[3-(4-methoxyphenyl)-4-oxo-1,2,3,4-tetrahydro-2-quinazolinyl]phenoxy}-N-phenylacetamide |

As used herein, “cells” refers to any animal cell, tissue, or whole organism, including but not limited to mammalian cells, e.g., bovine, rodent, e.g., mouse, rat, mink or hamster cells, equine, swine, caprine, ovine, feline, canine, simian or human cells.

As used herein, “agent” refers to any substance that has a desired biological activity. An “ER stress inhibitory agent” has detectable biological activity in inhibiting cell death or treating a disease, condition or injury associated with ER stress, in a host.

As used herein, “effective amount” refers to an amount of a composition that causes a detectable difference in an observable biological effect, for example, a statistically significant difference in such an effect, particularly an ER stress inhibitory activity. The detectable difference may result from a single substance in the composition, from a combination of substances in the composition, or from the combined effects of administration of more than one composition. For example, an “effective amount” of a composition comprising an ER stress inhibitory compound may refer to an amount of the composition that detectably inhibits cell death resulting from ER stress, or another desired effect, e.g., to reduce a symptom of ER stress, or to treat or prevent a disease, condition or injury associated with or resulting from ER stress or another disease or disorder, in a host. A combination of an ER stress inhibitory compound and another substance in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the compositions and methods of the present invention. Such organisms include, but are not limited to, “mammals,” including, but not limited to, humans, monkeys, dogs, cats, horses, rats, mice, etc. In the context of the invention, the term “subject” generally refers to an individual who will receive or who has received treatment (e.g., administration of a compound of the invention, and optionally one or more other agents) for cell death resulting from ER stress or an associated disease, condition or injury.

As used herein, “pharmaceutically acceptable salts” refer to derivatives of an ER stress inhibitory compound or other disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of an ER stress inhibitory compound or other compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Thomas J. Tucker, et al., J. Med. Chem. 37:2437-2444, 1994. A chiral compound of Formula I may also be directly synthesized using a chiral catalyst or a chiral ligand, e.g. Mark A. Huffman, et al., J. Org. Chem. 60:1590-1594, 1995.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)O—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

The alkenyl can optionally be substituted with one or more alkyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃), 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkenylidenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkylidenyl, alkenylidenyl, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Obviously, numerous modifications and variations of the present invention are possible in light of the above teachings. It is therefore to be understood that within the scope of the appended claims, the invention may be practiced otherwise than as specifically described herein.

### Medical Indications

The compounds of the present invention, which inhibit ER-stress-induced cell death, have use in the treatment of the following ER-stress-related diseases, conditions and injuries: neuronal disease, including but not limited to: familial Alzheimer's disease, Parkinson disease, Huntington disease (polyQ disease), spinobulbar muscular atrophy/Kennedy disease (polyQ disease), spinocerebellar ataxia 3/Machado-Joseph disease (polyQ disease), prion disease, amyotrophic lateral sclerosis, and GM1 gangliodosis; metabolic disease, including but not limited to: diabetes mellitus general, Wolcott-Rallison syndrome, Wolfran syndrome, type 2 diabetes mellitus, homocysteinemia, Zα1-antitrypsin deficiency inclusion body myopathy, and hereditary tyrosinemia type 1; ischemia injury; heart and circulatory system injury, including but not limited to: cardiac hypertrophy, hypoxic damage, and familial hypercholesterolemia; viral infection; atherosclerosis; bipolar disease; and Batten disease.

### Pharmaceutical Compositions

The compounds of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the invention can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of the compounds of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will be further described by the following non-limiting examples.

### Example 1

Compounds that block cell death induced specifically as a result of ER stress (and not other cell death pathways) would be useful for interrogating the underlying mechanisms, as well as for ascertaining in vivo in animal models when ER stress is the inciting event responsible for cell demise and tissue injury. The feasibility of this undertaking was recently demonstrated in a publication where a screen for chemical inhibitors of neuronal cell death induced by tunicamycin (an inhibitor of N-linked glycoslyation that induces ER stress) was performed, resulting in identified compounds that suppress protein phosphatases responsible for dephosphorylation of eIF2α on serine 51, thus increasing accumulation of phosphorylated eIF2α and providing protection from apoptosis induced by several inducers of ER stress (Boyce et al., Science, 307:935-939, 2005). Interestingly, the prototype compound characterized (Salubrinal) apparently is not an active site inhibitor of the phosphatase, but rather specifically disrupts complexes containing GADD35 and protein phosphatase-1 (PP1), thereby preventing GADD34-mediated targeting of PP1 onto substrate phospho-eIF2α.

We have devised an alternative screening assay for identification of compounds that block cell death induced by ER stress, and have screened a library of compounds, thereby validating this approach.

Because cell death linked to ER stress is a prominent feature of several neurological diseases, we focused on developing a primary chemical library screening assay utilizing neuronal cells. CSM14.1 is a rat striatal neuroprogenitor cell line that was established by immortalization using a temperature-sensitive variant of SV40 Large T antigen (Zhong et al., Proc. Natl. Acad. Sci. USA, 90:4533-4537, 1993; Haas and Wree, J. Anat., 201:61-69, 2002). At permissive temperature (optimal at 32° C.), the cells proliferate and can be easily expanded in standard culture media for high throughput screening (HTS) assays. When cultured at the non-permissive temperature of 39° C., large T antigen is inactive and the cells cease proliferating and differentiate to produce neurons with characteristics of mature dopaminergic neurons (Zhong et al., Proc. Natl. Acad. Sci. USA, 90:4533-4537, 1993; Haas and Wree, J. Anat., 201:61-69, 2002).

For convenience, and because transient reductions in temperature that might be associated with large screening experiments could restore Large T activity, we elected to develop our HTS using undifferentiated CSM14.1 cells, with the plan to then confirm hits using differentiated cells. For monitoring cell death, we used a commercially available bioluminescense reagent that determines intracellular ATP content, without requirement for complicated cell processing steps (ATPlite, Perkin Elmer). Thus, ATP was used as a surrogate indicator of cell survival for the primary assay. To trigger cell death using a stimulus known to induce ER stress, we selected thapsigargin (TG), a sesquiterpene lactone that irreversibly inhibits the Ca2⁺-ATPase of the ER (Jiang et al., Exp. Cell Res., 212:84-92, 1994; Tsukamoto and Kaneko, Cell Biol. Int., 17:969-970, 1993).

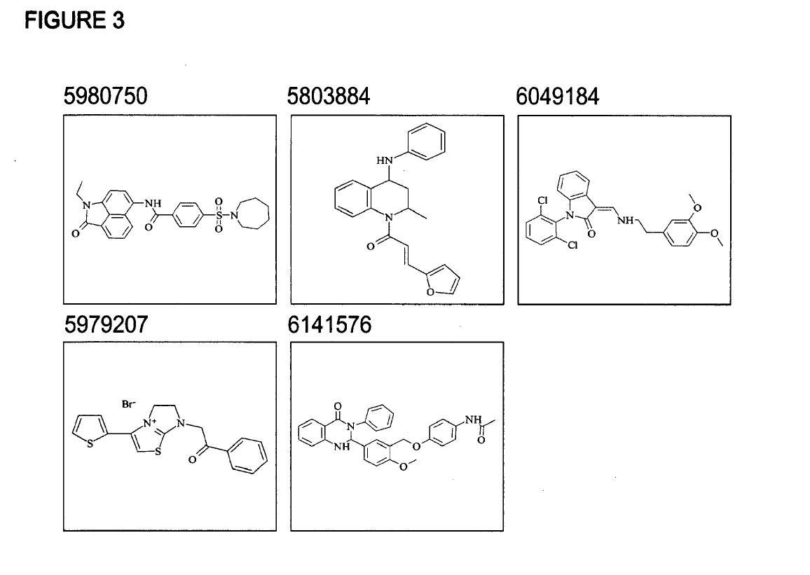

In pilot experiments, undifferentiated CSM14.1 cells were plated at various densities in wells of a 96 well plate, cultured overnight, then the ATP content of the cells was measured using the luminogenic ATPlite reagent (FIG. 4A). We also performed dose-response experiments for thapsigargin (FIG. 4B) by culturing CSM14.1 cells at 3,000 cells per well overnight, then treating the cells with various concentrations of TG or Sal and cultured for 18 hrs before determining ATP content (mean±SD; n=3) (FIG. 4B). Similarly, we performed dose response experiments for Salubrinal (Sal). Cells were plated and cultured as above, then treated with various concentrations of Salubrinal, followed two hrs later by 15 uM TG. Cells were cultured for 18 hrs, then ATP content was determined (mean±SD; n=3) (FIG. 4C). We determined that 3,000 cells per well (96 well, flat bottom, polystyrene plastic plates) provided an ample signal and that 15 μM thapsigargin (TG) resulted in approximately a 95% reduction in ATP per well, which was independently confirmed to correlate with ˜95% cell killing using vital dye exclusion assays. As a control, we employed Salubrinal, which was reported to block ER stress-induced cell death (Boyce et al., Science, 307:935-939, 2005). Salubrinal (100 μM) significantly protected against TG-induced cell death using undifferentiated CSM14.1 cells and measuring ATP as a surrogate for cell viability (FIG. 4).

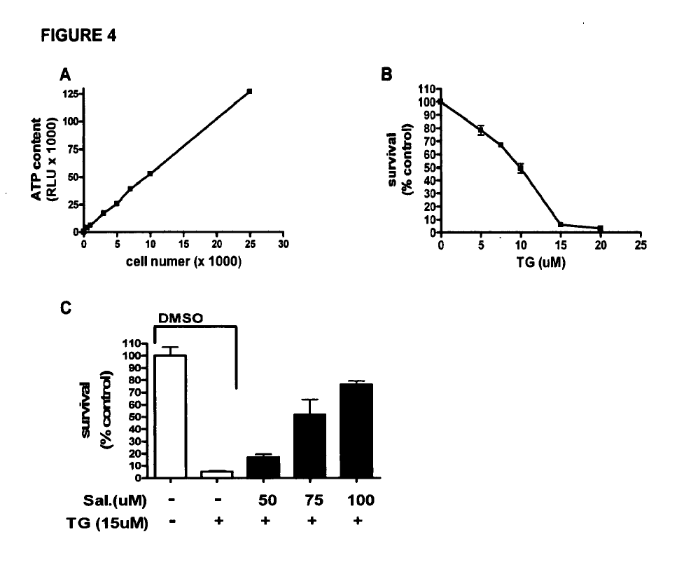

We compared undifferentiated and differentiated CSM14.1 neurons with respect to Salubrinal-mediated protection from TG-induced cell death. FIG. 5 shows that TG kills and Salubrinal protects CSM14.1 cells. Undifferentiated or differentiated CSM14.1 cells were plated at a density of 3,000 cells/well in 40 μL DMEM medium (containing 10% serum, 1 mM L-glutamine, and antibiotics) in 96 well flat-bottom microtiter plates composed of polystrene (Greiner Bio One, polystyrene, white wall, flat bottom, lumitrac, high binding). After overnight incubation at 32° C., DMSO (0.5% v/v) or 100 uM Salubrinal (Sal) in DMSO was delivered. After 2 hrs, 5 uL of a stock solution of 150 uM TG in DMEM medium containing 0.75% (v:v) DMSO to achieve a final concentration of approximately 15 uM. After culturing for 24 hrs, cellular ATP content was measured by addition of 20 uL per well of ATPlite solution (Perkin-Elmer), and then the plates were incubated for three minutes at room temperature, before reading with a Microplate Luminometer (BD Pharmingen Moonlight model 3096). The ATP content in cells treated with DMSO alone was used as a control for comparison, expressing results as a percentage relative to this control.

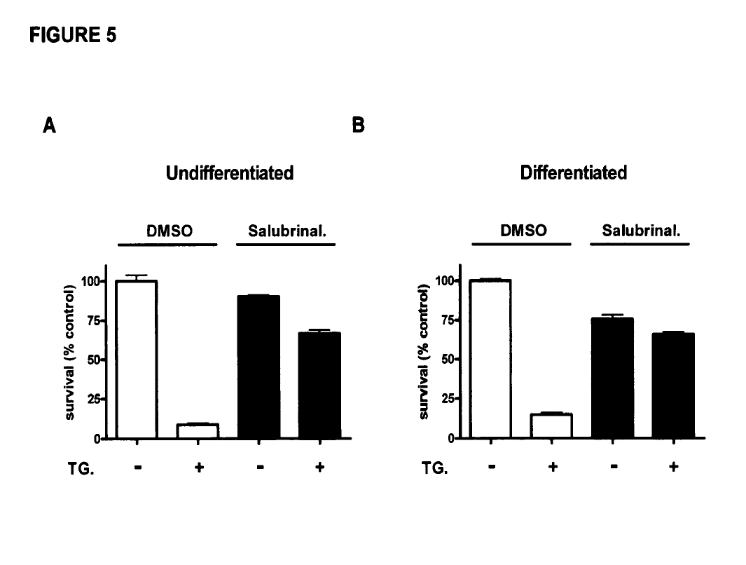

To determine the reproducibility of the ATP content assay, we prepared a 96-well plate in which half the wells received TG plus DMSO (assay minimum) and half received TG plus Salubrinal (assay maximum), then performed the ATP content studies using undifferentiated CSM14.1 cells (FIG. 6). CSM14.1 cells were cultured overnight at 3,000 cells per well in 96-well flat-bottom plates, then either DMSO (0.5% final concentration) or Salubrinal (100 μM) in DMSO (0.5% final concentration) was added to half the well (48 each). After two hrs, TG was added to all wells (15 μM final). Cells were cultured for 24 hrs, then the ATP content was measured using the ATPlite luminogenic assay. We then adapted this assay to the high-throughput environment, using a Biomex™ FX liquid handler for automated dispensing of assay components into microwells of 96-well plates. First, undifferentiated CSM14.1 cells were grown at 32° C. in DMEM medium supplemented with 10% fetal bovine serum (FBS), 1% penicillin/streptomycin, and 1% L-glutamine in 150 mm×25 mm polystyrene culture dishes to produce approximately 3×10⁶ cells. Second, CSM14.1 cells were recovered from cultures by trypsinization when at 80-90% confluence, and suspended at 7.5×10⁴ cells/mL in DMEM medium containing 2% FBS, 1 mM L-glutamine, and antibiotics 100 IU penicillin 100 μg/ml streptomycin. Third, the cell suspension was then delivered at 40 μL per well of 96-well plastic microtiter plates (Greiner Bio One, polystyrene, white wall, flat bottom, lumitrac, high binding, cat #655074), and the plates were cultured overnight at 32° C. in a humidified atmosphere in 95% air: 5% CO₂. Fourth, using the automated liquid handler, 5 μL of test compounds in 10% DMSO were added to wells in columns 2-11 of the 96-well plates (leaving columns 1 and 12 intact) to achieve an approximate final concentration of 15 μg/mL test compound and a final concentration of 1% DMSO. Fifth, 5 μL of 1 mM Salubrinal in a solution of 10% DMSO:90% DMEM was added to wells of column 1/rows A-D of each plate, while 5 μL of 10% DMSO:90% DMEM solution was added to wells corresponding to column 1/rows E-H and to all wells in column 12. Sixth, the plates were returned to culture. Seventh, after two hours, the automated liquid handler was used to dispense 5 μL per well of a stock solution of 150 μM TG in DMEM containing 0.75% DMSO, thus achieving a final concentration of ˜15 uM TG, in columns 1-11, thus leaving column 12 as a control for data comparison (1% DMSO/no TG). Eighth, the plates were returned to the incubator for 24 hrs. Ninth, using a liquid dispenser (Well Mate™ [Thermo-Fisher Scientific]), 20 μL of ATPlite solution was dispensed per well. Tenth, plates were read within 30 minutes using a Criterio-Analyst™ HT microplate recorder. Typically, signal:noise ratios were >7:1 and Z′ factors were >0.7, suggesting the assay method is suitable for HTS.

To assess the quality of screening data, the Z′ factor was calculated for each plate using an established formula (Zhang et al., J. Biomol. Screen. 4:67-73, 1999) and for the entire experiment, aggregating the min-control (DMSO only) and max-control (Salubrinal) results for all plates.

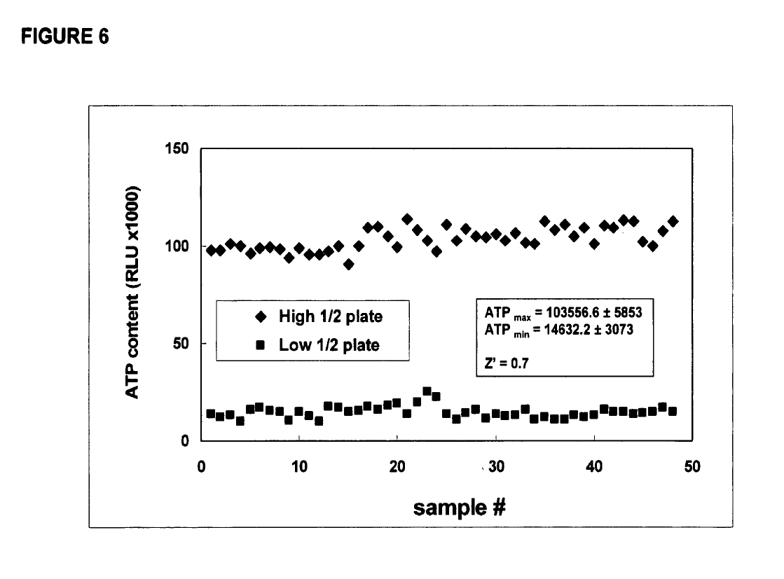

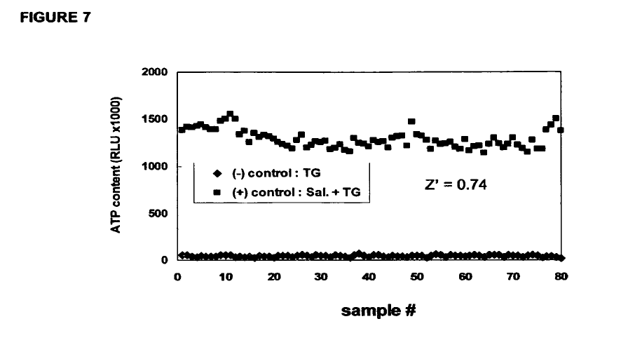

FIG. 7 shows an example of data from an assay quality control analysis from 20 plates, plotting the ATPlite results measured in relative luminescence units (RLU) (y-axis) for wells (x-axis) that received DMSO control and for wells that received Salubrinal, representing the minimum and maximum controls for the HTS assay. All wells received TG. For these 20 plates, the average signal: noise ratio was 31 and the Z′ factor was 0.74.

The basic method for screening a chemical library was as follows. Briefly on day 1, immortalized CSM14.1 cells were seeded as 3×10³ cells per well in white 96 well plates in 40 μl of DMEM supplemented with 2% FBS and antibiotics, followed by incubation overnight. On day 2, automatic liquid handler was used to add 5 μl of compounds to the plates (final 15 mg/ml in 1% DMSO). After 2 hours, cells are treated with thapsigargin (final 15 mM). 24 hours later, a luminescence assay is used to measure cytosolic ATP level. Cytosolic ATP activity is interpreted as relative survival rate comparing to non-treated control. To assess the quality of screening, a Z-prime (Z′) factor for each plate is calculated.

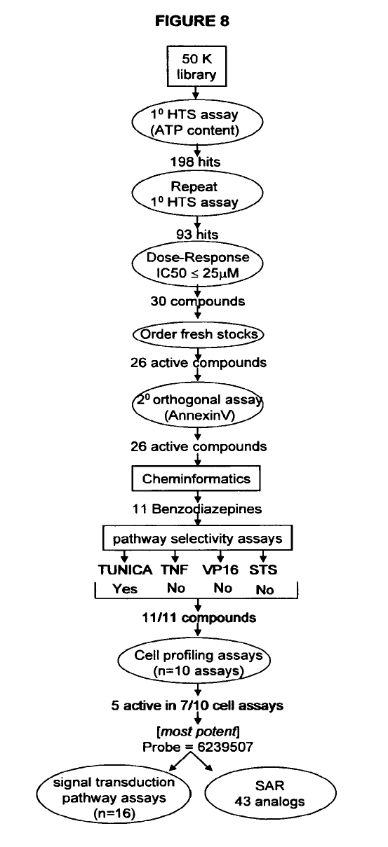

Using this assay, we screened an in-house library of 50,000 compounds (ChemBridge). Results for a typical plate are provided in FIG. 9A, showing raw data from a typical screening of an in-house library of 50,000 compounds (ChemBridge) showing one efficient hit compound (bold). CSM14.1 cells were treated with DMSO (1% final) (column 12), or with DMSO in combination with thapsigargin (column1 row E-H), or 100 mM salubrinal in combination with thapsigargin (column1 row A-D). The compound in the well at column 8, row G corresponds to a survival rate of 98.9%. Normalization of the raw data was accomplished by averaging the ATPlite signal (in Relative Luminescence Units) for column 12, representing cells that received 1% DMSO but no TG, and setting this value as 100. All other raw data values were then transformed by dividing by this average number (obtained for column 12) and multiplying by 100. FIG. 9B shows an example of screening results for a typical assay plate after normalization of data.

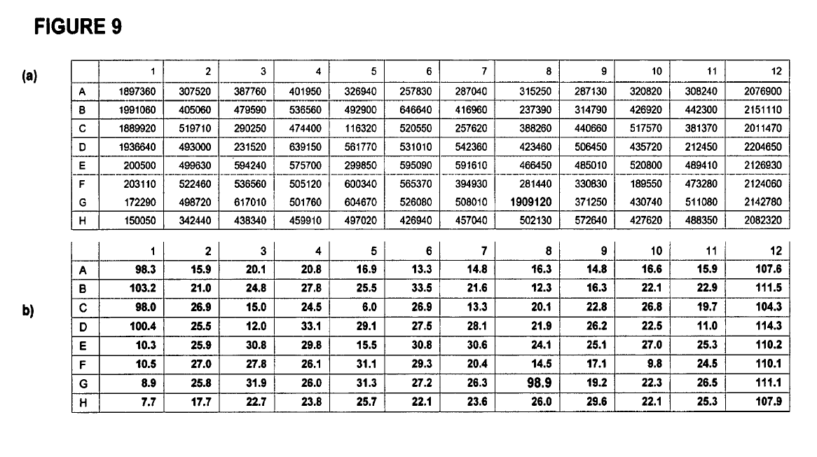

FIG. 10 is a graphical representation of an example of screening results after normalization of data. Relative ATP content (y-axis) is plotted against well number (1-96 [A1 to H12]) (x-axis). Wells A1 to D1 are assay maximum controls (received salubrinal+TG); Wells E1-H1 are assay minimum controls (received DMSO+TG); Wells A12-H12 (column 12) are normalization controls (received DMSO without TG). The average ATP content for wells A12-H12 was determined and used for normalizing data. The Z′ factor as calculated was 0.87 for this plate (if the Z′ factor is greater than 0.5 and less than 1.0, the assay is considered to be very stable). A hit compound is found in well G8 (arrow). To evaluate z prime factor, raw values of DMSO control samples were used as “control,” and those of thapsigargin-treated samples (column 1 row E-H) were used as ‘sample’ in the equation of z prime factor. If the Z value is over 0.5 and lower than 1.0, we consider the assay very stable.

From the screen of 50,000 compounds, 93 were identified that rescued CSM14.1 viability by >50% (Table 1, above). We then performed dose-response experiments using the same primary assay for these 93 hits, identifying 26 compounds that showed appropriate dose-response behavior with IC₅₀ (effective dose for rescuing 50% of the ATP content) <25 μM (Table 12):

TABLE 12
Compounds that rescue CSM14.1 cells from thapsigargin cell
death by >50% and have an IC₅₀ <25 μM

| | ChemBridge Compound |
|---|---|
| | ID Number |
| 1 | 6239507 |
| 2 | 6237735 |
| 3 | 5998734 |
| 4 | 6238475 |
| 5 | 6237877 |
| 6 | 5397372 |
| 7 | 6239538 |
| 8 | 5955734 |
| 9 | 6238767 |
| 10 | 6049448 |
| 11 | 5990041 |
| 12 | 5976228 |
| 13 | 5963958 |
| 14 | 5979207 |
| 15 | 5980750 |
| 16 | 5803884 |
| 17 | 6033352 |
| 18 | 6237973 |
| 19 | 6141576 |
| 20 | 5951613 |
| 21 | 6044673 |
| 22 | 5948365 |
| 23 | 6034674 |
| 24 | 6035098 |
| 25 | 5990137 |
| 26 | 6049184 |

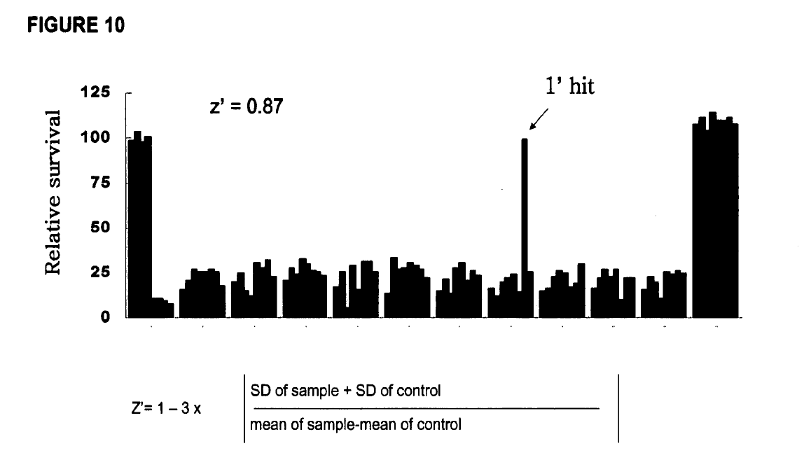

FIG. 11 shows the dose-dependent inhibition of ER stress-induced cell death by two hit compounds, along with two compounds that were discarded because of weak activity (C) or partial inhibition (D). Undifferentiated CSM 14.1 cells were treated with thapsigargin (15 μM) and with various concentrations of four of the compounds (A, B, C, D). The data are representative of three independent experiments.

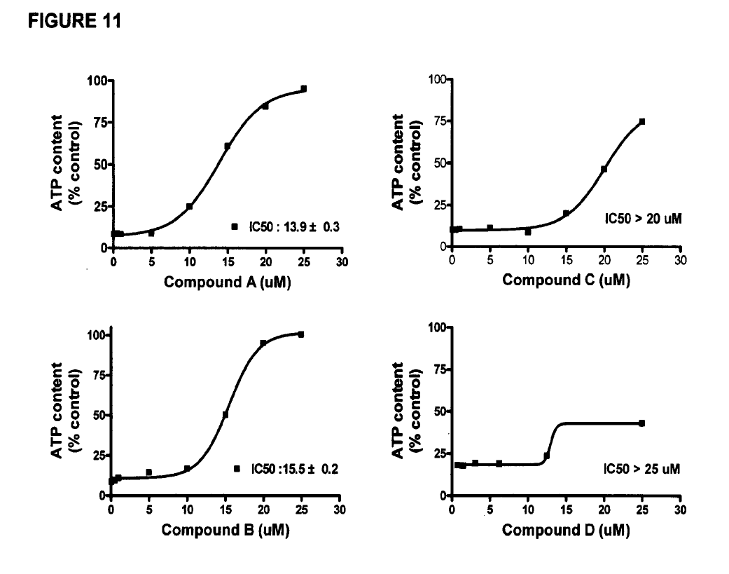

FIG. 12 shows that salubrinal inhibits thapsigargin-induced cell death less efficiently than our hit compounds. CSM 14.1 cells were plated at a density of 3×10³ cells per well in 96-well plates and incubated overnight. The indicated concentration of salubrinal was pre incubated with cells for two hours, followed by 7.5 mM thapsigargin treatment. 18 hours later, cell death rates were measured by the MTS assay. For the MTS assay, the CellTiter 96® Aqueous Non-radioactive Cell Proliferation Assay kit (Promega) was used. The reaction-ready solution was made according to the manufacturer's protocol, and each treated well of the 96-well plate was incubated with 20 μl of the reaction-ready solution. The plates were incubated humidified cell incubator at 37° C. with 5% CO₂ for two hours, and the absorbance of each well was read by an ELISA plate reader at 490 nm wavelength. To determine the background level of absorbance, the same volume of culture media (DMEM without cells) was incubated with MTS solution. The background value was subtracted from the value of each well. Background values from the control treatment (no TG, no Sal) wells were set as 100% survival, and the experimental wells' values were evaluated as the percentage of the control value.

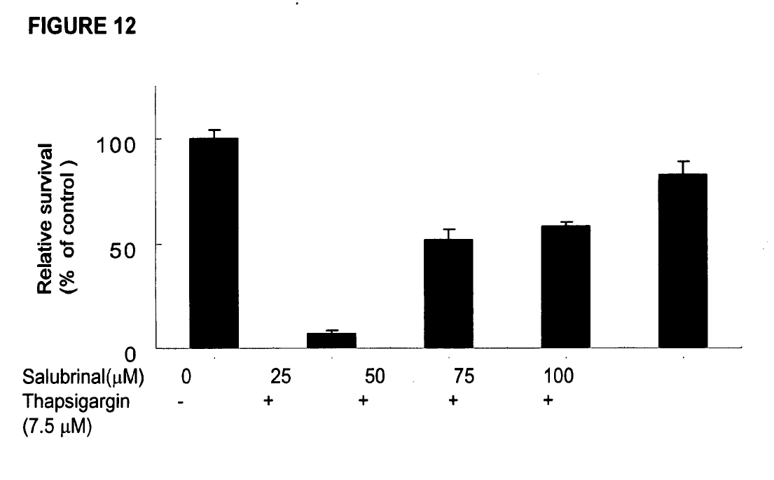

FIG. 13 compares the efficiency at which our hit compounds inhibit tunicamycin-induced cell death with salubrinal. CSM 14.1 cells were plated at a density of 3×10³ cells/well in 96-well plates and incubated overnight. Cells were pre-incubated with 25 μM of each compound or 100 μM salubrinal for two hours, followed by 10 mg/ml tunicamycin treatment. 72 hours later, cell death rates were measured by flow cytometry analysis. Annexin V-negative population was considered as survivors. In FIG. 13, the white column represents 0.5% DMSO control showing 24% of survival, and the gray column 100 μM salubrinal. Black columns are data from each compound (25 μM) (compound numbers refer to the compounds in Table 9). Although a 100 μm concentration of salubrinal is provides 75% inhibition of TG-induced cell death, several of the hit compounds identified in our screen provided equal or greater inhibition at a three-fold lower concentration.

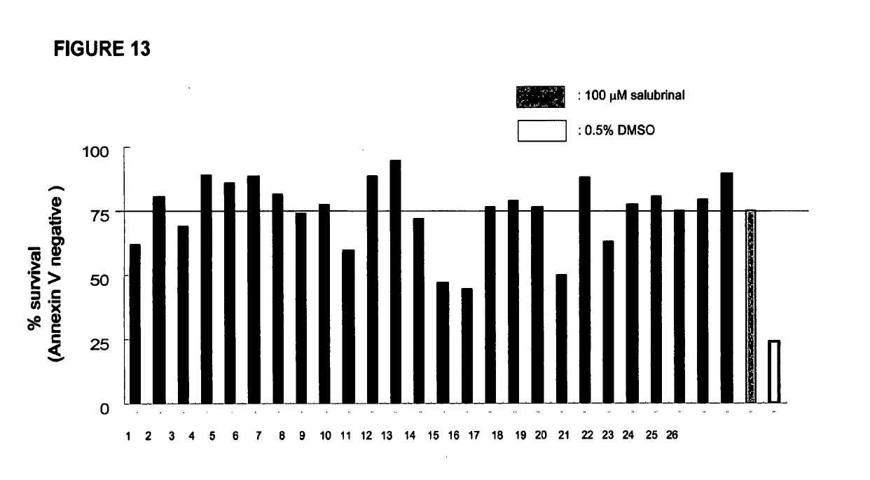

Of the 26 compounds shown in Table 9, 16 were subsequently confirmed to protect differentiated CSM14.1 cells from TG-induced cell death. For these assays, CSM14.1 cells were differentiated by culture in 2% FBS for 7 days at non-permissive temperature of 39° C. FIG. 14 shows a comparison of the cytoprotective activity of compounds using undifferentiated versus differentiated CSM14.1 neuronal cells. CSM14.1 cells were plated at 1,500 cells per well in 96-well plates, and cultured overnight at 32° C. (permissive temperature; FIG. 14, left) or at 39° C. (non-permissive temperature, FIG. 14, right) for 7 days. Various hit compounds were added at a 25 μM final concentration, followed two hrs later by TG at 15 μM final concentration. ATP content was measured, and data were expressed as a percentage of control cells treated only with 1% DMSO (mean±SD; n=3). Two percent FBS was used during differentiation, but all assays were performed in 10% FBS.

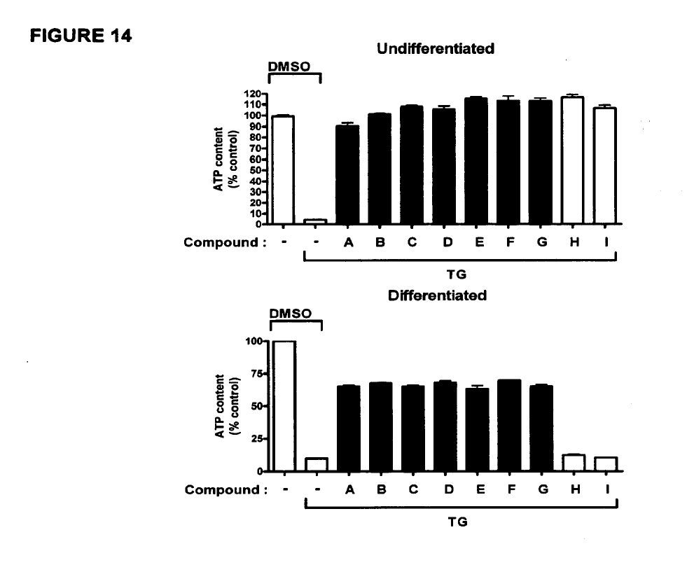

To explore whether compounds broadly protect cells of various lineages versus only neuronal cells, we tested all 26 hits for ability to rescue CSM14.1 (neuronal), and Jurkat (lymphoid), cell lines from cell death induced by TG. Of the 26 compounds tested with CSM14.1 and Jurkat, three showed protection in both cell lines. FIG. 15 shows an example of data comparing three of the 26 compounds for cytoprotective activity on CSM14.1 versus Jurkat cells. CSM 14.1 (FIG. 15, left) and Jurkat cells (FIG. 15, right) were cultured overnight at 3,000 cells per well or at 30,000 cells per well, respectively, in 96-well plates. Wells received DMSO alone or 25 μM compounds in DMSO, followed by treatment with or without TG (15 μM). After culturing for 24 hrs, ATP content was determined, expressing data as a percentage control relative to cells treated only with DMSO (mean±SD; n=3). The data reveal that one of the compounds protects CSM14.1 but not Jurkat cells from TG-induced cell death (as measured by the ATPlite assay). Because only three of the 26 compounds protected all three cell lineages, the assay employed here may have the ability to identify compounds with tissue-specific differences in activity—a property of considerable interest and utility. Alternatively, the compounds may detect species-specific differences, since CSM 14.1 cell are of rat origin, while HeLa and Jurkat are human.

Several alternative methods of assessing cell viability can be employed as secondary assays for confirming hits are truly cytoprotective and that they do not represent false-positives due to the peculiarities of the bioluminescent ATP assay. One method we have employed, for example, for confirming protection against TG-induced killing uses fluorochrome-conjugated annexin V staining with flow-cytometry analysis to evaluate the percentage cell viability by a method that is independent of the ATP content assay.

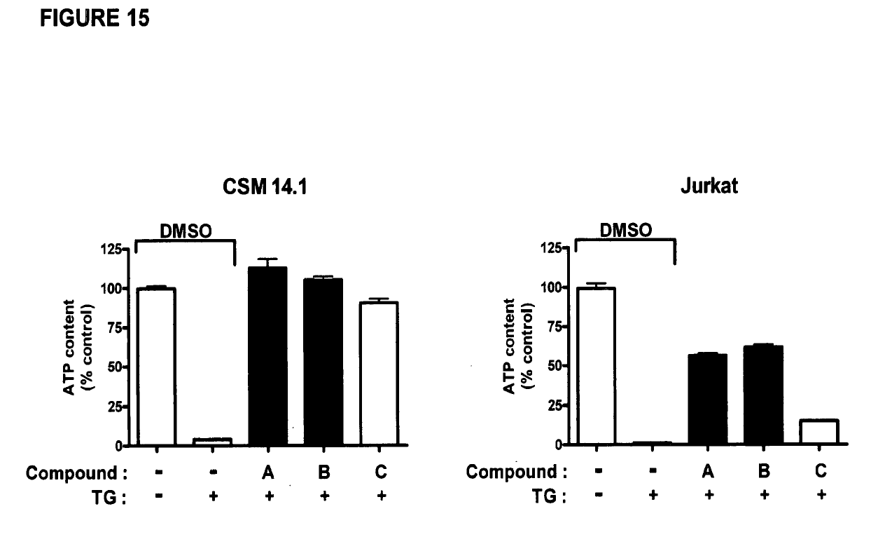

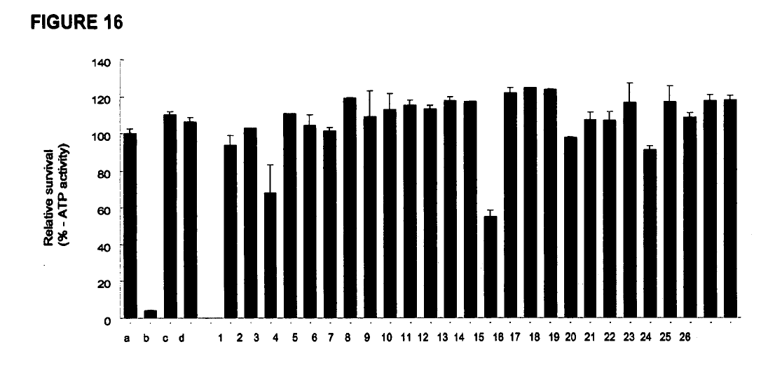

FIGS. 16 and 17 show the results of a secondary assay for evaluating the cytoprotective activity of the compounds. In FIG. 16, undifferentiated CSM14.1 cells were cultured at 10⁴ cells per well of 24-well plates (Greiner Bio One). The next day, DMSO (a, b) (1% final volume), 100 μM Salubrinal (c, d) or 25 μM of hit compounds (1% final DMSO) was added. After two hrs, 15 μM TG was added to all wells except a and c. A conventional ATP assay was performed to measure survival rate. In FIG. 17 undifferentiated CSM14.1 cells were cultured at 10⁴ cells per well of 24-well plates (Greiner Bio One). The next day, DMSO (a, b) (1% final volume), 100 μM Salubrinal (c, d) or 25 μM of hit compounds (1% final DMSO) was added. After two hrs, 15 μM TG was added to all wells except a and c. The plates were returned to culture for 24 hrs, then cells were recovered by trypsinization, transferred to 1.5 ml microcentrifuge tubes, and resuspended in 0.5 mL of Annexin V-binding solution containing 0.25 μg/mL Annexin V-FITC (Biovision) and propidium iodide. The percentage of annexin V-negative cells was determined by flow-cytometry (y-axis), using a FACSort instrument (Beckton-Dickinson). All 26 compounds protected against TG-induced cell death, as measured by annexin V staining, although two of the compounds (#3 and #14) were less active.

The selectivity of compounds with respect to suppression of cell death induced by ER stress was determined by treating undifferentiated CSM14.1 cells with a variety of agents that induce apoptosis via the ER stress pathway (thapsigargin, tunicamycin), the mitochondrial pathway (VP16) or the death receptor pathway (TNF+cycloheximide [CHX]). Of the 26 hit compounds tested, 19 reduced cell death induced by thapsigargin and tunicamycin but not VP16 or TNF/CHX.

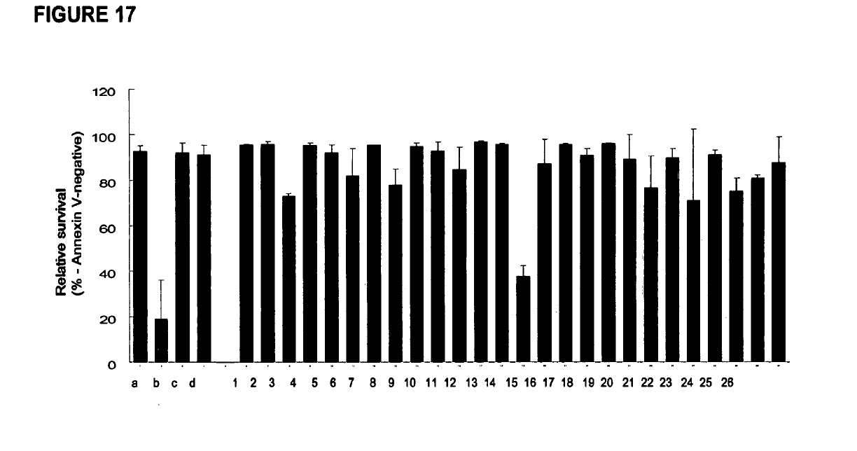

FIG. 18 shows an example of results for various hit compounds listed in Table 13.

TABLE 13
Benzodiazepine compound hits shown in FIG. 18

| Hit # | Chembridge ID | IC50 (μM) |
|---|---|---|
| 1 | 6239507 | 10.17 ± 1.621 |
| 2 | 6237735 | 12.5 ± 0.1859 |
| 4 | 6238475 | 12.5 ± 0.1695 |
| 5 | 6237877 | 12.5 ± 0.1695 |
| 7 | 6239538 | 12.5 ± 0.1766 |
| 9 | 6238767 | 12.5 ± 0.1769 |
| 10 | 6049448 | 23.73 ± 0.642 |
| 12 | 5976228 | 12.5 ± 0.1531 |
| 13 | 5963958 | 12.5 ± 0.1789 |
| 18 | 6237973 | 19.49 ± 0.1691 |
| 21 | 6044673 | 21.15 ± 0.3619 |

For the experiments shown in FIG. 18, undifferentiated CSM 14.1 cells were plated at 3,000 cells per well in 96-well plates (for the ATP assay) or at 1×10⁴ cells per well in 24-well plates (for flow cytometry). The next day, cells were treated with DMSO (0.5%) or hit compounds 25 μM of a compound with 0.5% DMSO final concentration) for two hours, followed by treatment with various cell death-inducing reagents, including 15 μM Thapsigargin (TG) for 24 hrs, 10 μg/mL tunicamycin (TU) for 72 hrs, 2.5 μM staurosporine (STS) for 24 hrs, 50 μM VP16 for 48 hrs, or 30 ng/mL TNF plus 10 μg/mL cyclohexamide (CHX) for 24 hrs. Cellular ATP content was measured for staurosporine samples and TNF/CHX samples, normalizing data relative to cells treated with DMSO alone (the control) and presenting as a percentage of control. For measuring cell death resulting from treatment with tunicamycin and VP16, flow cytometry was used. All assays were performed in triplicate (mean±SD).

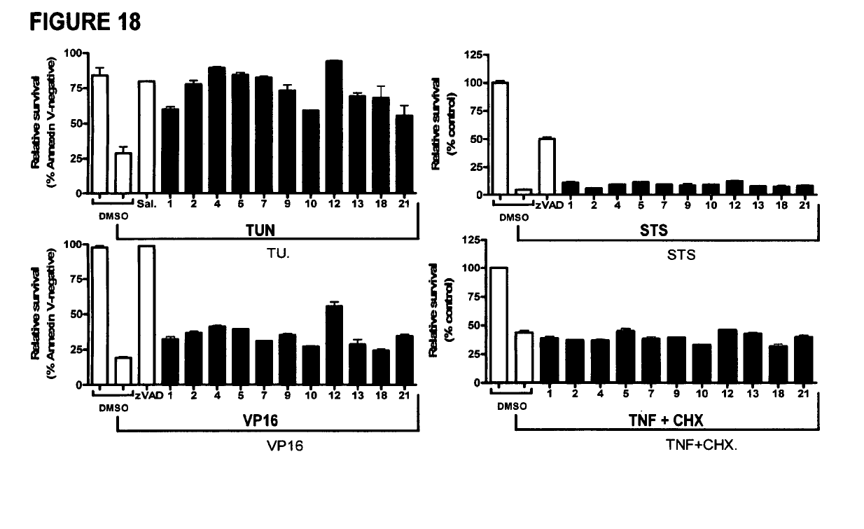

After confirming the pathway selectivity of compounds, additional downstream assays can be performed to map the specific signal transduction pathway inhibited by the compounds. In this regard, various antibody reagents are commercially available for assessing the status of the three major pathways known to be activated by ER stress: (1) PERK, (2) Ire1, and (3) ATF6 (Xu et al., J. Clinical Invest., 115:2656-2664, 2005). Immunoblotting experiments can be performed to assess the expression or phosphorylation (using phospho-specific antibodies) of marker proteins in these pathways. For example, as shown in FIG. 11, several of our hit compounds from the primary screen of an in-house library were found to suppress TG-induced phosphorylation of c-JUN and p38 MAPK, markers of the Ire1 pathway. The compounds tested in FIG. 19 are those listed in Table 9 above. CSM 14.1 cells were cultured with DMSO or with 25 μM of hit compounds for two hours, followed by treatment of thapsigargin (15 μM). Cell lysates were prepared and analyzed by SDS-PAGE/immunoblotting using antibodies specific for phospho-c-Jun, phospho-eIF2α, phospho-p38 MAPK, and tubulin (a loading control). CSM14.1 cells were cultured with either DMSO or one of the active compounds at 1, 5, and 10 μM, followed two hours later by 15 μM TG. After two hrs, cell lysates were prepared, normalized for protein content, and either analyzed by SDS-PAGE/immunoblotting using anti-p38-MAPK pan-reactive antibody or phospho-specific antibody with ECL-based detection, followed by densitometry analysis of x-ray films, normalizing phosphor-p38 MAPK relative to total p38 MAPK, or analyzed using a meso-scale instrument from MSD and a procedure in which total p38 MAPK is captured on plates, and the relative amounts of phosphorylated protein were determined using phospho-specific antibody (MSD catalog #K15112D1). The data shown in FIG. 19 suggest that these hit compounds act on the Ire1 pathway, which is known to trigger activation of the kinase Ask1, which in turn activates JNK and p38 MAPK. Suppression of p38 MAPK and c-Jun phosphorylation was dose-dependent, and could not be explained by a decline in total levels of these proteins. Moreover, compound-mediated inhibition of p38 MAPK phosphorylation in cells treated with TG was demonstrated by two independent methods: (1) immunoblotting using phospho-specific antibodies, and (2) using a plate-based method employing a meso-scale discovery instrument from MSD that uses SULFo-TAG(TM) labels that emit light upon electrochemical stimulation initiated at the electrode surfaces of MULTI SPOT microplates. Unlike Salubrinal, our compounds did not influence phosphorylation of eIF-2α, suggesting they do not act within the PERK pathway. Proteolytic processing of ATF6 was also not inhibited by our hit compounds. Thus, all of the hits we obtained from an in-house library of 50,000 compounds were mapped to the Ire1 pathway, and were found to suppress activation of JNK and p38MAPK. Screens of other libraries (such as the NIH compound collection) may yield cytoprotective compounds with different mechanisms.

Primary HTS Assay Protocol

Our primary HTS assay protocol is as follows.

1) Undifferentiated CSM14.1 cells are maintained at 32° C. in DMEM supplemented with 10% FBS, 1% penicillin/streptomycin, 1% L-glutamine (working concentration: 100 I.U. Penicillin/ml, 100 ug/ml streptomycin, 250 ng/ml Amphotericin: Media Tech) in 150 mm×25 mm polystyrene culture dishes (Falcon) to produce approximately 3×10⁶ cells/dish.

2) CSM14.1 cells are recovered from cultures by trypsinization when at 80-90% confluence, centrifuged at 400×g, and suspended at a density of 7.5×10° cells/mL in DMEM medium containing 2% FBS and the same concentration of antibiotics as in step 1.

3) The cell suspension is then delivered at 40 μL per well of 96-well plastic microtiter plates (Greiner Bio, polystyrene, white wall, flat-bottom, lumitrac, high binding), using a Well Mate liquid dispenser (Thermo Fisher Scientific), and the plates are cultured overnight at 32° C. in a humidified atmosphere in 95% air: 5% CO₂.

4) Library compounds are prepared at approximately 150 ug/mL in 10% DMSO plus 90% sterilized distilled water.

5) Using an automated liquid handler (Biomek™ FX liquid handler, Beckman Coulter), 5 μL of test compounds in 10% DMSO were added to wells in columns 2-11 of the 96-well plates (leaving columns 1 and 12 intact) to achieve an approximate final concentration of 15 μg/mL of test compound and a final concentration of 1.1% DMSO at this point.

6) Into wells of column 1/rows A-D of each plate, add 5 μL of 1 mM Salubrinal in a solution of 10% DMSO+90% DMEM (prepared by diluting 100 mM Salubrinal in DMSO 10 times by DMSO, then diluting this 10 mM Salubrinal in DMSO 10 times by same DMEM).

7) Into the wells of column 1/rows E-H and column 12/rows A-H, add 5 μL of 10% DMSO+90% DMEM solution.

8) Return plates to culture for two hours.

9) Dispense 5 μL per well of a stock solution of TG in DMEM (prep: 5 μl TG solution is composed of 0.0375 μl of 20 mM TG plus 4.9625 μl of DMEM containing 2% FBS and same concentration of antibiotics), thus achieving a final concentration of ˜15 μM TG, to columns 1-11, leaving column 12 as a control for data comparison (only DMSO/no TG). We use a ‘Well Mate’ liquid dispenser (Thermo Fisher Scientific).

10) To column 12, add 5 μL of DMEM (2% FBS and same concentration of antibiotics) containing 0.0375 μL of DMSO.

11) Return plates to the incubator for 24 hrs.

12) Approximately 30 minutes before use, ATP assay powder is dissolved into assay buffer (supplied by manufacturer) according to the manufacturer's protocol [Perkin-Elmer].

13) Dispense 20 μL of ATPlite solution per well, for example, using a ‘Well Mate’ dispenser (Thermo Fisher Scientific).

14) Within 30 minutes, measure luminescence. We used an Analyst™ HT (Molecular Device Corporation) with Criterio analysis software in the luminescence mode.

15) Relative Luminescence Units (RLU) recorded from the plate reader are imported into an EXCEL file. Each well's raw value was divided by average of raw values from all wells of column 12 (Max. control DMSO only/no TG) and multiplied by 100 to represent percentage relative to control.

For liquid dispensing in which cells (step 3), THS (step 9) or ATPlite solution (step 13), we used small nozzle tubing (Thermo Fischer Scientific). Before use, tubing was sterilized by 70% ethanol, and washed intensively with sterilized DW.

Secondary Assays

We perform compound conformation studies using differentiated CSM14.1 cells and other indicator cells, using ATP content as a surrogate indicator of cell viability, thus ascertaining which compounds display cytoprotective activity broadly versus narrowly. We also perform pathway selectivity analysis in which cell death is induced by agents know to trigger the ER stress pathway, mitochondrial pathway, or death receptor pathway, using ATP content as an end-point, thus determining which of the hit compounds are selective for the ER pathway. We also perform cell viability assays for hits that have EC50<25 uM and that show appropriate dose-response relations using alternative assays, such as annexin V staining as shown above or using colorimetric mitochondria-dependent dye reduction reagents such as MTT or XTT. Alternatively, or in addition, cell viability assays may be used. Finally, we map compounds to specific pathways known to be activated by ER stress using antibody-based methods, measuring phosphorylation of c-Jun, p38MAPK, and eIF2α, and measuring proteolysis of ATF6, as initial markers for interrogating compound mechanisms. The secondary assay protocols are as follows:

(a) Annexin-V Staining Viability Assay:

1) Undifferentiated CSM14.1 cells were cultured at 104 cells per well of 24-well plates (Greiner Bio one) in 400 μL of DMEM containing 2% FBS and antibiotics as described above.

2) The next day, DMSO (a, b) (1% final volume), 100 μM Salburinal (c, d) or 25 uM of hit compounds (1% final DMSO) was added. Briefly, 50 μL of DMEM containing 5 μL of DMSO, and 50 μL of DMEM containing 5 μL of 10 mM Salubrinal (or 2.5 mM compound) in DMSO was added for indicated wells.

3) After 2 hrs, 15 μM TG was added to all wells except a and c; 50 μL of DMEM containing 0.3754 of 20 mM TG in DMSO was added.

4) The plates were returned to culture for 24 hrs.

5) 24 hours later, all cells in wells were acquired by media transfer and trypsinization. All acquired cells were centrifuged in 1.5 mL microtube with DMEM-trypsin solution by 6,000 rpm for 2 minutes.

6) After aspiration of liquid, cells were washed with cold PBS smoothly. After centrifugation by 6,000 rpm, cells were resuspended in 500 uL of 1× Annexin V binding buffer (Biovision 1035-100) including 0.25 mg/mL Annexin V FITC (Biovision 1001-1000) and Propidium Iodide (50 ug/mL).

7) The percentage of annexin V-negative cells was determined by flow-cytometry (y-axis), using a FACSort analysis facility in Burnham (Beckton & Dickinson)

(b) Immunoblotting:

1) CSM14.1 cells were plated at a density of 2×105 cells/well at 6 well dish (Greiner Bio one) in DMEM containing 2% FBS and antibiotics.

2) After overnight incubation, cells were treated by DMSO (0.5%) or compounds (25 μM) for two hours, followed by TG (15 uM) treatment.

3) After two hours, cells were lyzed in 250 uL of lysis buffer and subjected to protein concentration decision, and to SDS PAGE/Western blotting using antibodies specific for phospho p38 MAPK (Cell signaling 9211), p38 MAPK (Santa Cruz-C20), phospho c-Jun (Cell signaling 9164), c-Jun (Santa Cruz-SC1694), phospho eIF2α (Cell signaling 3597) and α-tubulin.

(c) MSD Electrochemical Assays:

The same scheme employed for immunoblotting was used, except cells were lysed by lysis buffer (MSD Company). Half of cell lysate was used for Western blotting/densitometry analysis and the half for MSD plate-based assays. MSD assays were performed using the manufacturer's protocol.

1) Cells were lyzed using the supplied lysis buffer. Cell extracts were diluted in supplied dilution buffer, and quantified for 6 ug in 120 uL dilution buffer.

2) p38/p-p38 duplex plates (MSD company-Cat #K15112D-1) were blocked by supplied blocking buffer for 1 hour.

3) 120 μL of cell extracts were added to each well, and incubated overnight at room temperature with shaking.

4) After incubation, wells were washed 4 times with Tris wash buffer (supplied), incubated with detection antibody solution (supplied) for 1 hour, and washed 4 times with wash buffer again.

5) Finally, each well gained 150 uL of reading buffer (supplied), and the luminescence value was read by MSD Sector™ instrument.

6) The instrument showed luminescence value of p38 and phospho-p38 (p-p38). Value of p-p38 was divided by p-38 in each well. The control (DMSO treated, thapsigargin no treated) well's p-p38/p38 value was set as 1 for control, and other wells values were calculated by times of control. Finally, each value minus 1 was reported in this figure, because there was no back ground expression level of p-p38 in DMSO control. The graph was made based on each sample's ratio of ‘p-p38/p38’.

SAR Analysis.

In addition to verifying which hit compounds selectively block death induced by ER stress and mapping them preliminarily to one of the three known pathways triggered by ER stress (or to an unidentified pathway if none of the three known pathways are suppressed), SAR analysis is performed on selected hits, with the goal of advancing the potency and the selectivity of the compounds to “probe” status.

### Example 2

### Chemical Name of Chembridge Compound ID No. 5962123 and Commercial Availability

The chemical name of compound 5962123 is 6-(4-diethylaminophenyl)-9-phenyl-5,6,8,9,10,11-hexahydrobenzo[c][1,5]benzodiazepin-7-one. Compound 5962123 is available from ChemBridge. Recommended negative control compounds include ChemBridge 6075841 or 6048163. These benzodiazepines are inactive in the cell death assay used for primary screening and fail to suppress thapsigargin-induced phosphorylation of Jun.

Description of Biological Activity.

Compound 5962123 inhibits the thapsigargin (an inducer of ER stress)-induced death of both undifferentiated and differentiated rat neuronal cell line CSM14.1 with IC₅₀˜10 μM using two different indicators of cell viability: (a) ATP content assay, and (b) a flow cytometry-based assay for Annexin V staining. Compound 5962123 also inhibits cell death induced by tunicamycin (another inducer of ER stress) in CSM14.1 cells, but does not inhibit CSM14.1 cell death induced by TNF-a (plus cycloheximide), an agonist of the death receptor (extrinsic) cell death pathway or by either VP-16 or staurosporine (agonists of the mitochondrial cell death pathway), suggesting it is a selective inhibitor of ER stress-induced cell death (i.e., pathway-specific).

In addition to CSM14.1 cells, when tested at 25 μM, Compound 5962123 protected by >50% against thapsigargin-induced death of several tumor cell lines (HeLa human cervical cancer, SW1 melanoma cell, PPC1, human prostate cancer), mouse neural stem cell C17.2 (both differentiated [neuronal phenotype] and non-differentiated [stem cell phenotype]) as determined by ATP content assay, and primary rat cortical neurons as determined by microscopy assay measuring the percentage of NeuN-immunopositive cells with either normal or apoptotic nuclear morphology (Hoechst dye staining). However, compound 5962123 does not protect Jurkat human T-leukemia or either undifferentiated or differentiated (neuronal phenotype) PC12 rat pheochromocytoma cells from thapsigargin-induced cell death, as determined by an ATP content assay at 25 μM. In fact, compound 5962123 showed paradoxical cell death-promoting activity when tested on undifferentiated PC12 cells treated with thapsigargin.

Overall, while showing some cell type-selectivity, compound 5962123 is reasonably broad-spectrum in its cytoprotective activity, protecting 6 of 8 cell lines or cell types (primary neurons) tested.

Testing in thapsigargin-stimulated CSM 14.1 cells showed that compound 5962123 at 10 μM inhibits the UPR signaling pathway that results in phosphorylation of the JNK substrate c-Jun (measured by immunoblotting with phospho-specific antibody) and phosphorylation of p38MAPK, as determined by immunoblotting using phospho-specific antibodies and by a quantitative ELISA-based assay (MSD assay), but not the UPR-pathways involving PERK-mediated phosphorylation of elF2alpha (measured by immunoblotting with phosphospecific antibody), thapsigargin-induced expression of ATF4 (measured by immunoblotting), ATF6 proteolytic processing (assayed by immunoblotting to detect cleaved form), XBP1 mRNA splicing (assayed by RT-PCR to measure ratio of unspliced: spliced mRNA), Ire1 auto-phosphorylation (measured by in vitro kinase assay), ASK1 autophosphorylation (measured by in vitro kinase assay), thapsigargin-induced activation of ASK1 in cells (measured in vitro using a coupled kinase assay containing MKK6 and p38MAPK, to which ASK1 recovered from compound-treated cells by immunoprecipitation was added), or thapsigarin-induced expression of CHOP.

Compound 5962123 inhibited thapsigargin-stimulated dephosphorylation of ASK1 at serine 967 at 50 μM, measured in ASK1-transfected/thapsigargin-stimulated HEK293T cells by immunoblotting using phospho-specific antibodies, and it also increased 14-3-3 binding to ASK1, as determined by co-immunoprecipitation assay using the same transfected HEK293T cells stimulated with thapsigargin. Thus, while not directly inhibiting ASK1 kinase activity, these events are predicted to reduce ASK1 in vivo kinase activity. It is possible that compound 5962123 inhibits a protein phosphatase that regulates phosphorylation of Ser967.

Description of Secondary Screens.

The secondary screens used to characterize compound 5962123 (many of which were used to characterize all 11 benzodiazepines) are outlined above. Thirty-one secondary screens have been used to date to characterize compound 5962123. The compound is active with an IC₅₀˜10 μM as an inhibitor of thapsigargin-induced cell death of undifferentiated CSM 14.1 cells as measured by ATP content and as an inhibitor of tunicamycin-induced cell death of undifferentiated CSM14.1 cells as measured by the ATP content assay. The compound's activity against ER stress-induced cell death was confirmed by flow cytometric analysis, measuring annexin V staining of CSM14.1 cells treated with either thapsigargin or tunicamycin. When tested at 25 μM against undifferentiated CSM14.1 cells by the ATP content assay, compound 5962123 at 25 μM was not active against cell death induced by TNF-alpha plus cycloheximide, VP-16, and staurosporine. The compound's activity in neuronal cells was confirmed at 25 μM using differentiated rat neuronal CSM 14.1 cells treated with 10 μM thapsigargin using the ATP content assay, differentiated mouse neuronal C17.2 cells treated with thapsigargin using the ATP content assay, but not in differentiated rat pheochromocytoma PC12 cells treated with thapsigargin using the ATP content assay. The compound showed paradoxical cell death-promoting activity against thapsigargin-treated undifferentiated PC12 cells. Finally, compound 2878746 inhibits thapsigargin-induced cell death of rat primary cortical neurons (identified by staining with NeuN), as determined by counting apoptotic neurofilament (NeuN)-positive cells stained with the DNA-binding fluorochrome Hoechst dye to identify cells with condensed nuclear morphology indicative of apoptosis and evidence of neurite retraction.

Cytoprotective activity of compound 5962123 was also demonstrated in several types of non-neuronal human tumor cell lines treated with thapsigargin using the ATP content assay, including cervical carcinoma HeLa, human prostate cancer PPC-1, and human melanoma SW1 cells. The compound, however, was inactive against thapsigargin-treated Jurkat T-leukemia cells, as determined by the ATP content assay.

In terms of mechanism, the compound inhibits thapsigarin-induced phosphorylation of c-Jun and p38MAPK in CSM 14.1 cells, as determined by immunoblotting using phospho-specific antibodies (phospho-c-Jun Ser 63, and phosphor p38MAPK Thr180/Tyr182). Suppression of thapsigargin-induced phosphorylation of p38MAPK was also measured by a quantitative ELISA-methods, with IC₅₀ for p38MAPK phosphorylation estimated at <5 μM. In contrast, thapsigargin-induced expression of CHOP, expression of ATF4, proteolytic processing of ATF6, phosphorylation of eIF2α(Ser 51), auto-phosphorylation of Ire1a or auto-phosphorylation of ASK1 were not inhibited directly by CID-2878746 at concentrations up to 50 μM tested by in vitro kinase assay using p32-7-ATP substrate. Compound 5962123 also failed to inhibit cellular activation of ASK1, as determined by a coupled in vitro kinase assay containing purified MAPKK6 (MKK6/SKK3) and purified p38 MAPK, together with immunoprecipitated ASK1 derived from HEK293T cells that had been transfected with ASK1 plasmid and incubated with 100 μM compound plus 15 μg/mL Thapsigargin, prior to immunoprecipitating ASK1 and adding it to the couple assay. Thapsigargin-induced reductions in phosphorylation of ASK1 at the serine 967 site in ASK1 transfected 293T cells are inhibited by compound 5962123 at concentrations of 50-100 μM, as determined by immunoblotting using anti-phospho-specific (ser 967) antibody, but thapsigargin-induced changes in phosphorylation of ASK1 at ser 83 and thr 845 are not modulated by compound 5962123 at concentrations as high as 100 μM in ASK1-transfected HEK293T cells. Compound 5962123, at concentrations of 100 μM, also increases binding of ASK1 to 14-3-3 protein, as determined in a co-immunoprecipitation assay, using thapsigargin-stimulated, ASK1 transfected, HEK293T cells. Compound 5962123, at a concentration of 100 μM, did not affect activity of protein phosphatase 2B (Calcineurin) tested by an in vitro phosphatase assay using immunoprecipitated ASK1 (ser 967 site) as the substrate.

Chemistry Strategy Leading to Identification of Compound 5962123.

SAR analysis of compound 5962123 was performed, addressing three functionalities by analyzing data on 41 analogs, in addition to the SAR inherent in the primary screening data that demonstrated 11 active benzodiazepine hits. The assay used to compare the activity of compounds was the same as the primary HTS assay, in which undifferentiated CSM14.1 cells were challenged with thapsigargin and the cell viability was assessed using an ATP content assay. The potency data on the analogs are shown in Table 14 (R groups R1-R7 are substituents for the structure of Formula I), and from these data compound 5962123 was selected based on potency and cellular activity profile.

TABLE 14
Potency data for analogs in the benzodiazepinone
series of ER stress-active compounds.

| Com- | | | | | | | | |
|---|---|---|---|---|---|---|---|---|
| pound | R1 | R2 | R3 | R4 | R5 | R6 | R7 | IC50 (μM) |
| 6239507 | OMe | OMe | | | | OPh | | 13.89 ± 0.2596: |
| | | | | | | | | 10.17 ± 1.621 |
| 6237735 | OMe | OMe | CF₃ | | | | | 17.16 ± 0.4705: |
| | | | | | | | | 12.5 ± 0.1859 |
| 6238475 | OMe | OMe | | | | CF₃ | | 18.81 ± 0.1566: |
| | | | | | | | | 12.5 ± 0.1695 |
| 6237877 | OMe | OMe | | | O—n-Pr | | | 16.26 ± 0.4393: |
| | | | | | | | | 12.5 ± 0.1695 |
| 6239538 | OMe | OMe | | | | Br | | 15.48 ± 0.2427: |
| | | | | | | | | 12.5 ± 0.1766 |
| 6238767 | OMe | | Me | | | Me | | 13.31 ± 1.1719: |
| | | | | | | | | 12.5 ± 0.1769 |
| 6049448 | OMe | | | Cl | OH | OEt | | 24.83 ± 2.031: |
| | | | | | | | | 23.73 ± 0.642 |
| 5963958 | | | | | SMe | | | 18.55 ± 9.14: |
| | | | | | | | | 12.5 ± 0.1789 |
| 6237973 | | | Cl | | | | F | 22.58 ± 0.6804: |
| | | | | | | | | 19.49 ± 0.1691 |
| 6044673 | OMe | | | | | OEt | | 20.15 ± 0.7806: |
| | | | | | | | | 21.15 ± 0.3619 |
| 6047795 | Cl | | | | Me | | | 10.95 ± 1.042 |
| 6049070 | Cl | | | | OMe | | | 12.5 ± 0.1302 |
| 6047998 | Cl | | O—i-Pr | | | | | 8.087 ± 0.8322 |
| 5962123 | | | | | NEt₂ | | | 7.499 ± 0.8572 |
| 5969327 | OMe | | | | | Cl | | 21.73 ± 1.070 |
| 6065973 | | | Cl | | | | | >200 |
| 6067345 | OMe | | | | NAc | | | >100 |
| 6079090 | OMe | | | | F | F | | 21.67 ± 0.2472 |
| 6076307 | OMe | OMe | Cl | | | | | 41.7 ± 0.9159 |
| 5969590 | OMe | | | | NEt₂ | | | 11.43 ± 1.486 |
| 6049861 | OMe | | | | i-Pr | | | 10.3 ± 1.284 |
| 6366991 | | | 1-Naphthyl | | | | | 22.18 ± 2.797 |
| 6043556 | OMe | | O—i-Pr | | | | | 24.02 ± 2.311 |
| 6048163 | OMe | | | | | OH | | >200 |
| 6045744 | OMe | | | | OH | | | >200 |
| 6240043 | OMe | OMe | Nitro | | | | | 39.61 ± 1.99 |
| 6069474 | | | | | OMe | | | 25 ± 0.1221 |
| 6045627 | OMe | | 1-Naphthyl | | | | | 13.36 ± 1.884 |
| 6066511 | | | O—n-Bu | | | | | 19.23 ± 2.16 |
| 6048306 | OMe | | | Br | OH | OEt | | 18.22 ± 1.925 |
| 6044487 | OMe | | Cl | | | | F | 15.27 ± 1.505 |
| 6241186 | OMe | | F | | | | F | 25 ± 0.1749 |
| 6079349 | OMe | | Cl | | | | Cl | 31.48 ± 2.843 |
| 6239945 | OMe | OMe | | | Ph | | | 21.77 ± 2.974 |
| 6105705 | OMe | OMe | | | Me | | | 25 ± 0.1691 |
| 6075841 | OMe | OMe | 4-pyridyl replaces Ph | | | | | >200 |
| 6369841 | OMe | OMe | Me | | | | | 36.45 ± 2.193 |
| K813-0010 | OMe | OMe | | | | CH₃ | | 24.91 ± 0.8326 |
| 5991-0620 | OEt | OMe | Cl | | | F | | >200 |
| 3192-5300 | OMe | OMe | | | | OH | | 53.46 ± 3.454 |

Synthetic Pathway for Making Compound 5962123.

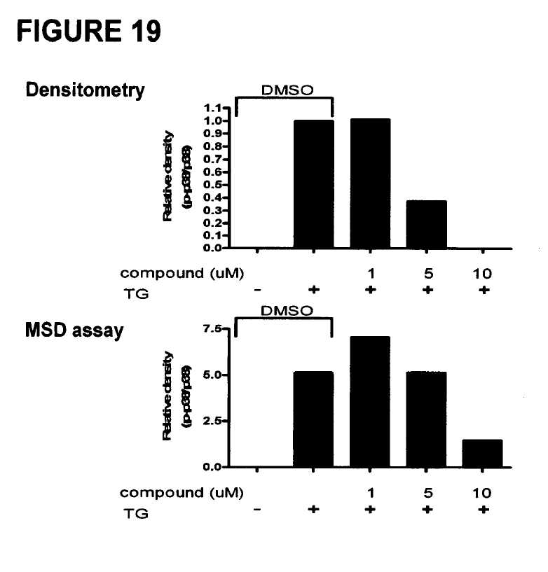

Compound 5962123 was resynthesized (FIG. 20). The analytical data and biological activity were identical to the purchased compound, thus confirming its structure and potency. Analytical data indicate the presence of all four possible stereoisomers in both the commercial and synthetic samples. In addition, another compound, MLS-0292126, was synthesized via the same route and showed an IC₅₀ of 16.5 μM.

Known Properties

A summary of the properties of compound 5962123 is provided in Table 15 below.

TABLE 15
Cryoprotective activity against various cell death stimuli assessed using CSM 14.1 cells

| Stimulus | Thapsigargin | Tunicamycin | VP-16 | TNFα + CHX | Staurosporine |
|---|---|---|---|---|---|
| Readout | ATP & Annexin | Annexin | Annexin | ATP & Annexin | ATP & Annexin |
| Cell type | CSM 14.1 | CSM 14.1 | CSM 14.1 | CSM 14.1 | CSM 14.1 |
| Result | + | + | − | − | − |

In Table 15 above, + indicates >50% rescue of cell viability relative to untreated cells not exposed to thapsigargin at compound concentration of ≦25 μM; ATP-ATPlite assay measuring ATP content of cells; Annexin V staining involves measuring the percent FITC-annexin V-positive cells as determined by flow cytometry.

TABLE 16
Cytoprotective activity against other cell lines

| | Stimulus | | | | | | | | |
|---|---|---|---|---|---|---|---|---|---|
| | Thapsigargin | | | | | | | | |
| | Readout | | | | | | | | |
| | ATP content | | | | | | | | |
| | Cell type | | | | | | | | |
| | CSM | | | | | | | | |
| | 14.1 | | | | | | PC12 | | C17.2 |
| | diff | HeLa | PPC1 | SW1 | Jurkat | PC12 | diff | C17.2 | diff |
| Result | + | + | + | + | − | E | − | + | + |

In Table 16 above, + indicates >50% rescue of cell viability relative to untreated cells not exposed to thapsigargin at compound concentration of ≦25 μM; thapsigargin concentration was 10 μM for C17.2 cells, 15 μM for all others; E indicates compound enhanced thapsigargin-induced death; diff=differentiated. For PC12, cells were stimulated with 20 ng/ml NGF for five days; for C17.2, cells were stimulated with serum reduction, and 1% N2 incubation for three days; for CSM14.1, cells were cultured at 39° C. for 5-7 days.

Properties of Compound 2878746.

The solubility of the compound 5962123 in dimethylsulfoxide (DMSO) is excellent at concentrations of 25 mM. For adding the compound to culture media, at least 0.2% (v/v) final DMSO concentration was needed to avoid producing a visible cloudy precipitate. At concentrations exceeding 25 mM, the compound in DMSO shows a yellow color. Negative control compounds 6048163 and 6075841 show similar solubility as 6239507 in DMSO. At a concentration of 25 mM, compound 6048163 showed a light yellow color, while compound 6075841 was colorless.

### Example 3

The potency data for various compounds of Group 2-1 and Group 2-2 were obtained as described above and are provided in Tables 17 and 18 respectively (R groups are substituents for the structure of Formulae II and III):

TABLE 17
Potency data for ER stress-active compounds of Group 2-1.

| Com- | | | | | | | | |
|---|---|---|---|---|---|---|---|---|
| pound | R1 | R2 | R3 | R4 | R5 | R6 | R7 | IC₅₀ (μM)* |
| 5998734 | | | OMe | | | Ph | | 20.02 ± 0.4928, |
| | | | | | | | | 14.63 ± 26.56 |
| 5955734 | | OMe | | OMe | | Ph | | 23.8, |
| | | | | | | | | 16.53 ± 1.467 |
| 5990041 | Br | | | OMe | | Ph | | 12.85 ± 0.3183, |
| | | | | | | | | 14.25 ± 1.204 |
| 6035098 | | | OMe | OMe | | Ph | | 19.75 ± 0.7206, |
| | | | | | | | | 16.63 ± 2.032 |
| 5990137 | OMe | | | OMe | | Ph | | 17.52 ± 0.6747, |
| | | | | | | | | 13.86 ± 1.059 |
| 6033487 | OMe | OH | OMe | | OMe | OMe | | >>200 |
| 5990076 | | OEt | | | | Ph | | >>200 |
| 5397316 | | | | OMe | OMe | OMe | OMe | >>200 |
| 5935965 | | N (R2 | | | OMe | OMe | OMe | >>200 |
| | | carbon | | | | | | |
| | | displaced) | | | | | | |
| 5951294 | | | Cl | | OMe | OMe | | >>200 |
| 5996087 | | | | OMe | | Ph | | 25 < IC50 < 50 |
| 6036165 | | | F | | OMe | OMe | | >>200 |
| 5952061 | OMe | OMe | OMe | | Cl | | | >>200 |
| 5998646 | OMe | OMe | | OMe | | Ph | | 35.57 ± 4.284 |
| 6033233 | OMe | OMe | OMe | | OMe | OMe | | >>200 |

*The IC₅₀ value in bold is from a second assay.

TABLE 18
Potency data for ER stress-active compounds of Group 2-2.

| Compound | R | IC₅₀ (μM)* |
|---|---|---|
| 5397372 | 3,4,5-methoxyphenyl | 18.89 ± 0.5683, |
| | | 13.47 ± 2.024 |
| 6033352 | 3,4-methoxy | 13.43 ± 0.2476, |
| | | 13.77 ± 0.5476 |
| 6034674 | p-nitrophenyl | 15.01 ± 0.4953, |
| | | 11.53 ± 0.5013 |
| 5951613 | 2-thiophene | 16.74 ± 0.1577, |
| | | 12.72 ± 1.106 |

*The IC₅₀ value in bold is from a second assay.

Note that the IC50 for compound 5948365 was determined to be 19.54±0.1769.

### Example 4

CSM 14.1 cells were cultured with DMSO or with 25 μM of hit compounds for two hours followed by treatment with thapsigargin (15 μM). Cell lysates were prepared and analyzed by SDS-PAGE/immunoblotting using antibodies specific for: c-Jun, phosphor-c-Jun (ser 73), eIF2α, phosphor-eIF2α (ser 51), p38 MAPK, phosphor-p38 MAPK (Thr180/Tyr182), ATF-6, CHOP and tubulin (loading control). ER stress-induced activation of C-Jun and p38 MAPK is suppressed by the 11 hit compounds.

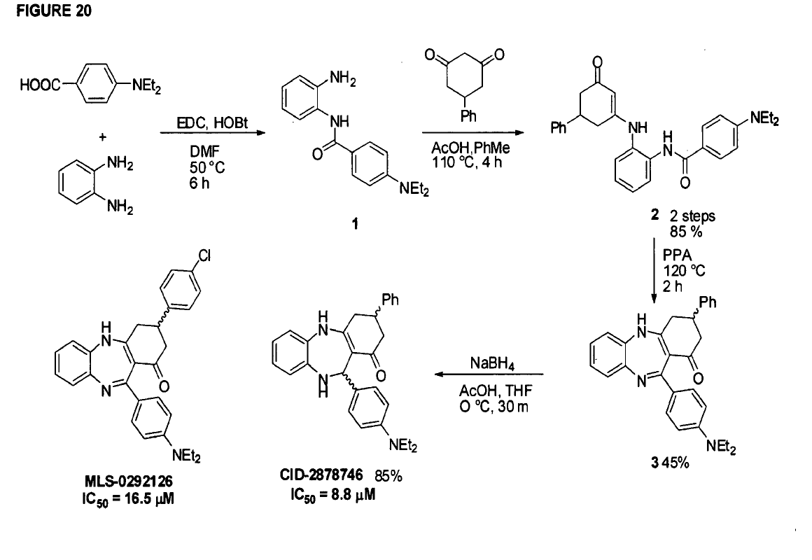

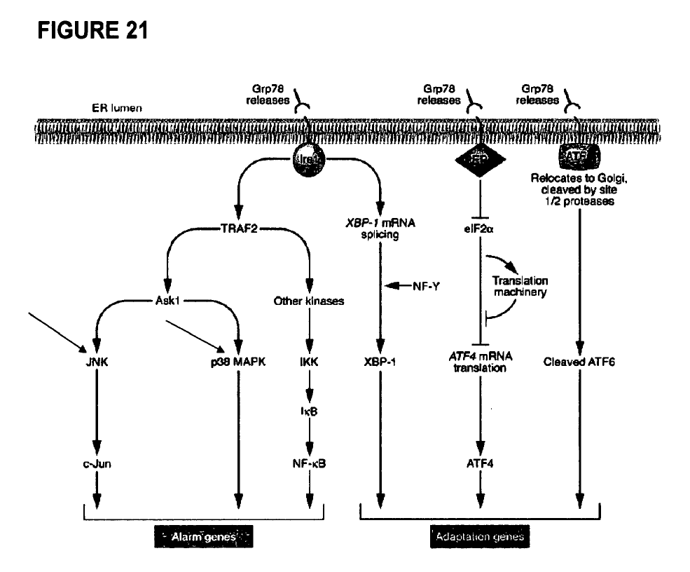

C-Jun and p38 MAPK work downstream of the Ire1 pathway (see FIG. 21), we tested whether one of our hit compounds (6239507) inhibits autophosphorylation of any of kinases in this Ire1-ASK1-JNK/p38 MAPK pathway. FIG. 22 shows the results of in vitro kinase assays using compound 6239507. An Ire1 autophosphorylation assay was performed. Immunoprecipitated Ire1 was incubated with DMSO (2%), 50 μM compound 6239507, or the positive control staurosporine (20 μM; STS) for 20 minutes at 30° C. followed by chilling on ice. 0.5 μCi of ³²P-γ-ATP was added to each tube and incubated at 30° C. for the indicated times. Kinase reactions were finished by adding sample buffer. Incorporation values of ³²P-γ-ATP were evaluated by a scintillation counter. An ASK1-MKK6-p38 coupled assay was also performed. Immunoprecipitated ASK1 was mixed with 1 μg MKK6 (Millipore) and 1 μg p38 MAPK (Sigma). Kinases were incubated with 2% DMSO, 50 μM compound 6239507, or 20 μM staurosporine (STS) in one tube for 20 minutes at 30° C. followed by ice chilling. 0.5 μCi of ³²P-γ-ATP was added to each tube and incubated at 30° C. for the indicated times. Kinase reactions were finished by adding sample buffer. Incorporation values of ³²P-γ-ATP were evaluated by a scintillation counter. C-Jun activation by purified Jnk-1 and Jnk-2 (Millipore) was confirmed with the same procedures. The results show that hit compound 6239507 does not modulate Ire1's autophosphorylation activity, Ask1's activity with respect to downstream kinases MKK6 and p38 MAPK, or JNK's activity on c-Jun.

Experiments were also performed to determine whether compound 6239507 enhances phosphorylation of ASK1 at Ser 967 before and after ER stress induction (i.e., thapsigargin treatment). 293T cells were transfected with pcDNA-ASK1-HA. One day later, cells were incubated with DMSO (0.4%) or 100 μM compound 6239507 (#1) for two hours. Then cells were treated with thapsigargin (20 μM) for the indicated times. Cell extracts were prepared by lysis buffer and were subjected to immunoblotting with anti-phospho ASK1 Ser967 antibody or anti HA antibody. The relative density of phosphor ser967 bands were calculated by imageJ software (mean±SD). Compound 6239507 was found to enhance phosphorylation of ASK1 at Ser 967 before and after ER stress induction.

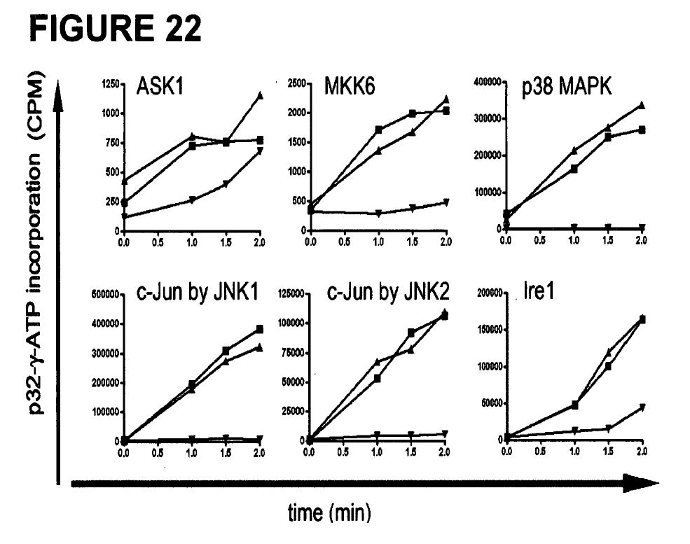

The ser 967 site of ASK1 is known to down-regulate ASK1 activity by phosphorylation (Goldman et al., J. Biol. Chem. 279:10442-10449, 2004) via 14-3-3 binding. We tested whether our hit compounds enhance phosphorylation only of ser 967 or also additional phosphorylation sites. 293T cells were transfected with pcDNA-ASK1-HA. One day later, cells were incubated with DMSO (0.4%) or 100 μM compound 6239507 (#1) for two hours. Cell extracts were prepared using lysis buffer and were subjected to immunoblotting using anti-phospho ASK1 antibodies or anti HA antibody as indicated. The relative density of each phosphorylated ASK band was calculated by imageJ software. The compounds were compared in activity against thapsigargin-induced cell death. 293T cells were transfected with pcDNA-ASK1-HA and pEBG-GST-14-3-3. One day later cells were incubated with DMSO (0.4%) or 100 μM of the indicated compound for two hours. Then cells were treated with thapsigargin (20 μM) for the indicated time. Cell extracts were prepared using lysis buffer, and 14-3-3 proteins were immunoprecipitated with glutathione S transferase 4B sepharose beads. ASK1 protein binding with 14-3-3 was visualized by immunoblotting using anti-HA antibody. Anti-phospho ASK1 (ser967) antibody was used to detect phosphorylation of ASK1 at each time point. As shown in FIG. 23, our hit compounds (marked as 1, 2, 9, 10, and 12) enhanced phosphorylation of ser 967 only, and not ser 83 or thr 845. TP 14 is another hit compound which has different structure from 1, 2, 9, 10 and 12. 6048163 is a compound that shares the same structural backbone with those hit compounds, but is inactive in cell protection (FIG. 23B). Compound 6239507 (indicated as #1 in FIGS. 23A and B) inhibits dissociation of 14-3-3 from ASK1 after thapsigargin treatment in a phosphorylation-dependent manner.

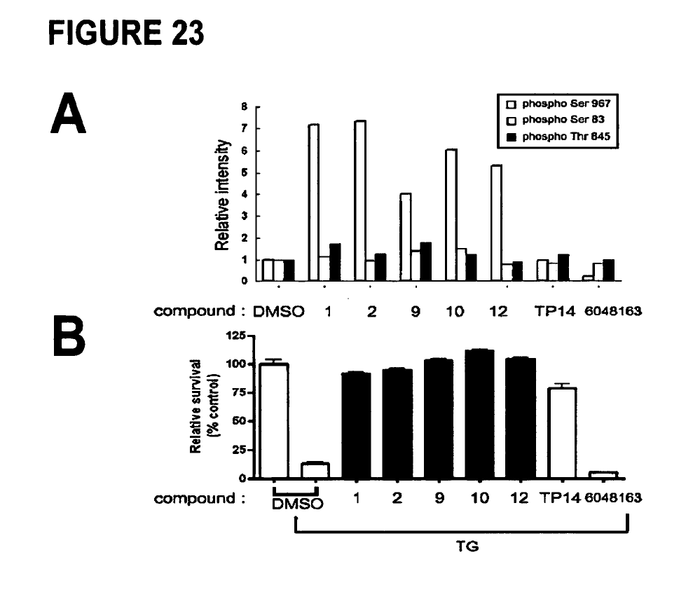

FIG. 24, shows our hypothesis about this mechanism. It is possible that the hit benzodiazepine compounds are inhibitors of ASK1 ser967 dephosphorylation. Thus, the compounds inhibit dissociation of 14-3-3 from ASK1, rendering ASK1 inactive.

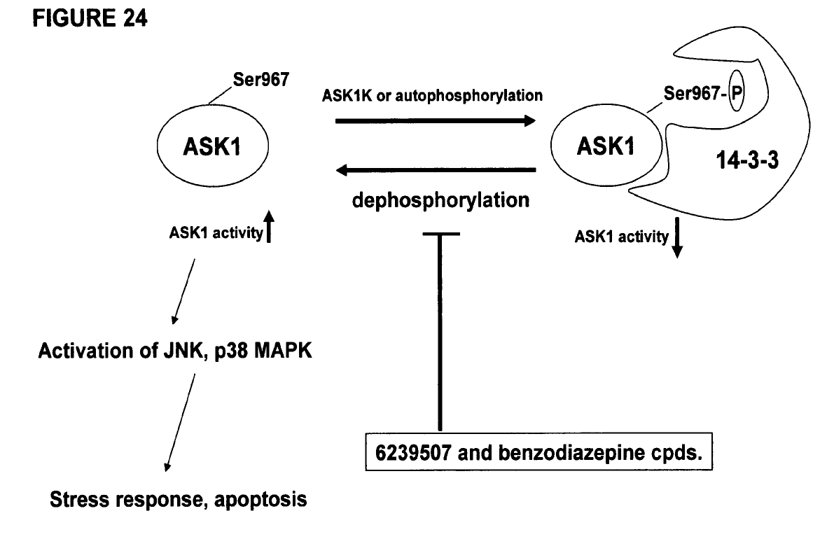

FIG. 25 shows that compound 6239507 can inhibit ER stress-induced cell death in primary mouse neuronal cells. Primary cortical neuron cells were prepared from the midbrain of mice. After 14 days of maturation, the cells were preincubated with DMSO (0.2%) or 25 μM of compound 6239507 for two hours. The cells were then treated with thapsigargin (TG) for 24 hours. Cells were fixed with an aldehyde solution and subjected to immunostaining with NeuN and MAP2 antibody for staining the neuronal body and axon network. Hoechst dye was used to stain nuclei. Fluorescent microscopy was used to show the loss of the axon network by thapsigargin. Cells showing a condensed nucleus and shrunken neuritis were considered as dead to evaluate cell death.

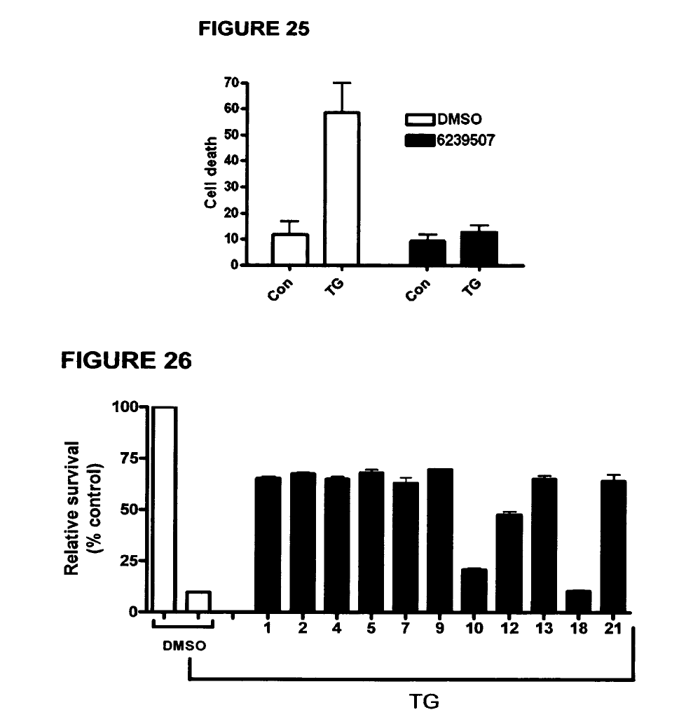

FIG. 26 shows relative survival for CSM14.1 cells treated with various hit compounds. CSM14.1 cells were plated at 1,500 cells per well in 96-well plates and cultured at 39° C. (non-permissive temperature) for 7 days. Hit compounds were added to a final concentration of 25 μM followed two hour later by thapsigargin at a final concentration of 15 μM. ATP content was measured and data were expressed as a percentage of control cells treated with only 1% DMSO.

We observed a dose-dependent decrease of c-Jun phosphorylation following treatment with compounds 6237877 and 6237735. CSM cells were treated with two compounds at increasing doses. Pre-incubation time, thapsigargin treatment, cell extract preparation and immunoblotting protocols were as described previously. The dose-dependent decrease of c-Jun phosphorylation was confirmed by anti-phospho-c-Jun (ser73) antibody.

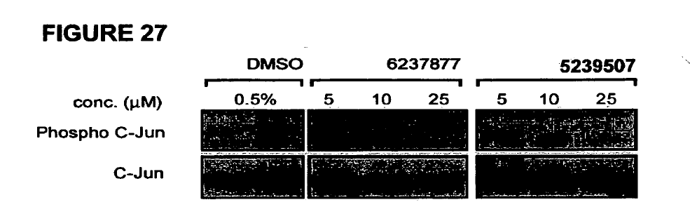

FIG. 27 shows that ER stress inhibitory compounds inhibit thapsigargin-induced markers of the Ire1 pathway. CSM 14.1 cells were cultured with DMSO or with the indicated compounds at 1 μM, 5 μM, and 10 μM, followed by treatment with thapsigargin (15 μM). After two hours, cell lysates were prepared, normalized for protein content, and either analyzed by SDS-PAGE/immunoblotting using anti-p38 MAPK pan-reactive antibody or phosphor-specific antibody with ECL-based detection, followed by densitometry analysis of x-ray films, normalizing phospho-p38 MAPK relative to total p38 MAPK (FIG. 27, top), or analyzed using a meso-scale instrument from MSD and a procedure in which total p38 MAPK is captured on plates, and the relative amounts of phosphorylated protein are determined suing phosphor-specific antibody (MSD catalog #K15112D1 (FIG. 27, bottom).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1-40. (canceled)

41. A method of inhibiting death of a mammalian cell resulting from endoplasmic reticulum stress comprising treating the cell with a composition comprising an effective amount of a compound that inhibits death of mammalian cells resulting from endoplasmic reticulum stress induced by thapsigargin.

42. A method of treating a disease, condition or injury of a mammal associated with endoplasmic reticulum stress comprising administering to a mammal in need thereof a composition comprising an effective amount of a compound that inhibits death of mammalian cells resulting from endoplasmic reticulum stress induced by thapsigargin.

43. The method of claim 42 wherein the disease, condition or injury is selected from the group consisting of neuronal disease, metabolic disease, ischemia injury, heart and circulatory system injury, viral infection; atherosclerosis, bipolar disease, and Batten disease.

44. The method of claim 43 wherein the neuronal disease is selected from the group consisting of familial Alzheimer's disease, Parkinson disease, Huntington disease, spinobulbar muscular atrophy/Kennedy disease, spinocerebellar ataxia 3/Machado-Joseph disease, prion disease, amyotrophic lateral sclerosis, and GM1 gangliodosis.

45. The method of claim 43 wherein the metabolic disease is selected from the group consisting of diabetes mellitus general, Wolcott-Rallison syndrome, Wolfran syndrome, type 2 diabetes mellitus, homocysteinemia, Zα1-antitrypsin deficiency inclusion body myopathy, and hereditary tyrosinemia type 1.

46. The method of claim 43 wherein the heart and circulatory system injury is selected from the group consisting of cardiac hypertrophy, hypoxic damage, and familial hypercholesterolemia.

47. The method of claim 42 wherein the mammal is a human.

48. The method of claim 41 wherein the mammalian cells are CSM 14.1 rat striatal neuroprogenitor cells.

49. The method of claim 41 wherein the composition inhibits death of the cells by about 50 percent or more.

50. The method of claim 41 wherein the composition has an IC₅₀ of about 25 μM or less.

51. The method of claim 41 wherein the composition inhibits death of CSM14.1 rat striatal neuroprogenitor cells by about 50 percent or more and has an IC₅₀ of about 25 μM or less.

52. The method of claim 42 wherein the composition comprises a compound selected from the group consisting of
1-(diphenylmethyl)-4-[(2-phenylcyclopropyl)carbonyl]piperazine hydrochloride,
9-ethyl-3-{[4-(3,4,5-trimethoxybenzoyl)-1-piperazinyl]methyl}-9H-carbazole,
4-{[4-[(2-bromo-4-meth-ylphenyl)amino]-6-(4-morpholinyl)-1,3,5-triazin-2-yl]oxy}-3-methoxybenzaldehyde semicarbazone,
2-{4-[4-(diphenylmethyl)-1-piperazinyl]-2-butyn-1-yl}-1H-isoindole-1,3(2H)-dione,
1-[3-(2-furyl)acryloyl]-2-methyl-N-phenyl-1,2,3,4-tetrahydro-4-quinolinamine,
N-[1,3-benzodi-oxol-5-yl(8-hydroxy-7-quinolinyl)methyl]acetamide,
1-[(4-{[4-(2-chlorophenyl)-1-piperazinyl]-carbonyl}phenyl)ethynyl]cyclohexanol,
diethyl 4-{3-[(4-fluorobenzoyl)amino]phenyl}-2,6-dimethyl-3,5-pyridinedicarboxylate,
N-2-biphenylyl-3-[(3,5-dimethoxybenzoyl)hydrazono]butan-amide,
N-[[4-(dimethylamino)phenyl](8-hydroxy-7-quinolinyl)methyl]propanamide,
4-(2,7-dimethylimidazo[1,2-a]pyridin-3-yl)-N-(4-ethoxyphenyl)-1,3-thiazol-2-amine hydrobromide,
1-(1-naphthylmethyl)-4-(4-nitrobenzoyl)piperazine,
(2-methoxydibenzo[b,d]furan-3-yl)(4-pyridin-ylmethyl)amine,
1-(diphenylacetyl)-4-(2-nitrobenzyl)piperazine oxalate,
9-ethyl-3-{[4-(2-thienylcarbonyl)-1-piperazinyl]methyl}-9H-carbazole oxalate,
3-(2-methoxy-1-naphthyl)-6-(trifluoromethyl)[1,2,4]triazolo[4,3-a]pyridine,
1-[3-(benzyloxy)-4-methoxybenzyl]-4-(4-nitro-benzoyl)piperazine,
1-(diphenylacetyl)-4-(2-methylbenzyl)piperazine,
1-(4-biphenylylcarbonyl)-4-(2,4-dimethoxybenzyl)piperazine,
1-(4-pyridinyl)-3-(2-thienyl)-3-thioxo-1-propanone,
11-[4-(methylthio)phenyl]-3-phenyl-2,3,4,5,10,11-hexahydro-1H-dibenzo[b,e][1,4]diazepin-1-one,
10-benzoyl-3-phenyl-11-(3-pyridinyl)-2,3,4,5,10,11-hexahydro-1H-dibenzo[b,e][1,4]diazepin-1-one,
11-{3-[(4-fluorophenoxy)methyl]-4-methoxyphenyl}-3,3-dimethyl-2,3,4,5,10,11-hexahydro-1H-dibenzo[b,e][1,4]diazepin-1-one,
11-(4-biphenylyl)-3,3-dimethyl-2,3,4,5,10,11-hexahydro-1H-dibenzo[b,e][1,4]diazepin-1-one,
7-(2-oxo-2-phenylethyl)-3-(2-thienyl)-6,7-dihydro-5H-imidazo-[2,1-b][1,3]thiazol-4-ium bromide,
4-(1-azepanylsulfonyl)-N-(1-ethyl-2-oxo-1,2-dihydrobenzo-[cd]indol-6-yl)benzamide,
2-[2-(4-methoxyphenyl)-1-phenylvinyl]-1H-benzimidazole,
2-(1,3-benzodioxol-5-yl)-N-(2-ethoxybenzyl)-N-(4-pyridinylmethyl)ethanamine,
7-benzoyl-11-(4-chlorophenyl)-2,3,4,5,10,11-hexahydro-1H-dibenzo[b,e][1,4]diazepin-1-one,
1-(4-biphenylyl-carbonyl)-4-(5-bromo-2-methoxybenzyl)piperazine oxalate,
1-(4-biphenylylcarbonyl)-4-(2,5-dimethoxybenzyl)piperazine,
1-(4-bromophenyl)-5-(2-thienylmethylene)-2,4,6(1H,3H,5H)-pyrimidinetrione,
1-(4-biphenylylcarbonyl)-4-(3-methoxybenzyl)piperazine,
2-[2-(2-methyl-1H-indol-3-yl)vinyl]-3-(3-nitrophenyl)-4(3H)-quinazolinone,
5-(4-chlorophenyl)-N-[4-(2-furoyl-amino)-3-methylphenyl]-2-furamide,
3-{[4-(3,4-dimethoxybenzoyl)-1-piperazinyl]methyl}-9-ethyl-9H-carbazole oxalate,
1-[3-(benzyloxy)-4-methoxybenzyl]-4-[(4-chlorophenoxy)acetyl]-piperazine,
9-ethyl-3-{[4-(4-nitrobenzoyl)-1-piperazinyl]methyl}-9H-carbazole,
1-(4-biphenylyl-carbonyl)-4-(2,3-dimethoxybenzyl)piperazine,
1-(4-nitrobenzoyl)-4-(3-phenoxybenzyl)-piperazine,
1-(4-biphenylylcarbonyl)-4-(4-chlorobenzyl)piperazine,
1-benzyl-4-(4-methoxy-benzoyl)-3,3-dimethyl-3,4-dihydro-2(1H)-quinoxalinone,
3,4-dimethoxybenzaldehyde O-{2-[4-(diphenylmethyl)-1-piperazinyl]-2-oxoethyl}oxime,
11-[4-(benzyloxy)phenyl]-3-(4-methoxy-phenyl)-2,3,4,5,10,11-hexahydro-1H-dibenzo[b,e][1,4]diazepin-1-one,
11-(4-chloro-3-nitro-phenyl)-3-(4-methoxyphenyl)-2,3,4,5,10,11-hexahydro-1H-dibenzo[b,e][1,4]diazepin-1-one,
N-1-naphthyltetrahydro-3a′H-dispiro[cyclohexane-1,2′-bis[1,3]dioxolo[4,5-b:4′,5′-d]pyran-7′,1″-cyclohexane]-5′-carboxamide,
11-(3-ethoxyphenyl)-3-(4-methoxyphenyl)-2,3,4,5,10,11-hexa-hydro-1H-dibenzo[b,e][1,4]diazepin-1-one,
11-(3-bromo-4-methoxyphenyl)-3-(4-methoxy-phenyl)-2,3,4,5,10,11-hexahydro-1H-dibenzo[b,e][1,4]diazepin-1-one,
N-[2-(2-methoxyphen-oxy)ethyl]-4-phenoxybenzamide,
3-(4-methoxyphenyl)-11-(2-methylphenyl)-2,3,4,5,10,11-hexahydro-1H-dibenzo[b,e][1,4]diazepin-1-one,
11-(2-chlorophenyl)-3-(4-methoxyphenyl)-2,3,4,5,10,11-hexahydro-1H-dibenzo[b,e][1,4]diazepin-1-one,
11-(3-bromo-5-ethoxy-4-hydroxy-phenyl)-3-(4-methoxyphenyl)-2,3,4,5,10,11-hexahydro-1H-dibenzo[b,e][1,4]diazepin-1-one,
3-(4-methoxyphenyl)-11-(3-methylphenyl)-2,3,4,5,10,11-hexahydro-1H-dibenzo[b,e][1,4]diazepin-1-one,
butyl 4-(2-furyl)-2-methyl-5-oxo-7-phenyl-1,4,5,6,7,8-hexahydro-3-quinolinecarboxylate,
1-(2,6-dichlorophenyl)-3-({[2-(3,4-dimethoxyphenyl)ethyl]amino}methylene)-1,3-dihydro-2H-indol-2-one,
11-(3-chloro-5-ethoxy-4-hydroxyphenyl)-3-(4-methoxyphenyl)-2,3,4,5,10,11-hexa-hydro-1H-dibenzo[b,e][1,4]diazepin-1-one,
6-(1H-1,2,3-benzotriazol-1-yl)-3,5-diphenyl-2-cyclo-hexen-1-one,
N-[1-(1H-benzimidazol-2-yl)-2-(2-thienyl)vinyl]-4-methylbenzamide,
2,2-diphenyl-N-[4-(4-pyridinylmethyl)phenyl]acetamide,
7-benzoyl-11-[4-(dimethylamino)phenyl]-2,3,4,5,10,11-hexahydro-1H-dibenzo[b,e][1,4]diazepin-1-one,
4-({4-[chloro(difluoro)methoxy]-phenyl}amino)-4-oxo-2-butenoic acid,
11-(5-bromo-2-thienyl)-3-(4-methoxyphenyl)-2,3,4,5,10,11-hexahydro-1H-dibenzo[b,e][1,4]diazepin-1-one,
3-{[2-(1H-indol-3-yl)ethyl]-amino}-5-phenyl-2-cyclohexen-1-one,
11-(4-methylphenyl)-3-phenyl-2,3,4,5,10,11-hexahydro-1H-dibenzo[b,e][1,4]diazepin-1-one,
methyl 4-[2-(benzyloxy)phenyl]-7-(3,4-dimethoxyphenyl)-2-methyl-5-oxo-1,4,5,6,7,8-hexahydro-3-quinolinecarboxylate,
11-(3-bromophenyl)-3-phenyl-2,3,4,5,10,11-hexahydro-1H-dibenzo[b,e][1,4]diazepin-1-one,
3-(1,3-benzodioxol-5-yl)-11-[4-(trifluoromethyl)phenyl]-2,3,4,5,10,11-hexahydro-1H-dibenzo[b,e][1,4]diazepin-1-one,
3-(3,4-dimethoxyphenyl)-11-[4-(methylthio)phenyl]-2,3,4,5,10,11-hexahydro-1H-dibenzo[b,e][1,4]-diazepin-1-one,
N,N-diethyl-4-{[(4-methoxyphenyl)(phenylsulfonyl)amino]methyl}benzamide,
7-benzoyl-3-phenyl-11-(3-pyridinyl)-2,3,4,5,10,11-hexahydro-1H-dibenzo[b,e][1,4]diazepin-1-one,
7-benzoyl-11-(4-hydroxyphenyl)-3-phenyl-2,3,4,5,10,11-hexahydro-1H-dibenzo[b,e][1,4]-diazepin-1-one,
N-(tert-butyl)-3,5-bis[(phenylacetyl)amino]benzamide,
2,4-dichloro-N-(3-{[(2-naphthyloxy)acetyl]amino}phenyl)benzamide,
N,N′-(sulfonyldi-3,1-phenylene)di(2-thiophene-carboxamide), 1-[(3-chloro-1-benzothien-2-yl)carbonyl]-4-(2-ethoxyphenyl)piperazine,
N-(2,4-dimethoxyphenyl)-3-methyl-2-(4-methylphenyl)-4-quinolinecarboxamide,
N-(4-{[2-methoxy-5-(4-oxo-3-phenyl-1,2,3,4-tetrahydro-2-quinazolinyl)benzyl]oxy}phenyl)acetamide,
3-(3,4-dimeth-oxyphenyl)-11-[2-(trifluoromethyl)phenyl]-2,3,4,5,10,11-hexahydro-1H-dibenzo[b,e][1,4]-diazepin-1-one,
3-(3,4-dimethoxyphenyl)-11-(4-propoxyphenyl)-2,3,4,5,10,11-hexahydro-1H-dibenzo[b,e][1,4]diazepin-1-one,
11-(2-chloro-6-fluorophenyl)-3-phenyl-2,3,4,5,10,11-hexa-hydro-1H-dibenzo[b,e][1,4]diazepin-1-one,
3-(3,4-dimethoxyphenyl)-11-(4-ethylphenyl)-2,3,4,5,10,11-hexahydro-1H-dibenzo[b,e][1,4]diazepin-1-one,
11-[4-(benzyloxy)-3-methoxy-phenyl]-3-(3,4-dimethoxyphenyl)-2,3,4,5,10,11-hexahydro-1H-dibenzo[b,e][1,4]diazepin-1-one,
tetrahydro-2-furanylmethyl 4-(9-ethyl-9H-carbazol-3-yl)-2-methyl-5-oxo-1,4,5,6,7,8-hexahydro-3-quinolinecarboxylate,
3-(3,4-dimethoxyphenyl)-11-[3-(trifluoromethyl)phenyl]-2,3,4,5,10,11-hexahydro-1H-dibenzo[b,e][1,4]diazepin-1-one,
11-(2,5-dimethylphenyl)-3-(4-methoxyphenyl)-2,3,4,5,10,11-hexahydro-1H-dibenzo[b,e][1,4]diazepin-1-one,
11-(4-chloro-3-nitrophenyl)-3-phenyl-2,3,4,5,10,11-hexahydro-1H-dibenzo[b,e][1,4]diazepin-1-one,
7-benzoyl-11-(4-nitro-phenyl)-2,3,4,5,10,11-hexahydro-1H-dibenzo[b,e][1,4]diazepin-1-one,
3-(3,4-dimethoxyphenyl)-11-(3-phenoxyphenyl)-2,3,4,5,10,11-hexahydro-1H-dibenzo[b,e][1,4]diazepin-1-one,
11-(3-bromophenyl)-3-(3,4-dimethoxyphenyl)-2,3,4,5,10,11-hexahydro-1H-dibenzo[b,e][1,4]diazepin-1-one,
11-(1,3-benzodioxol-5-yl)-8-methyl-3-phenyl-2,3,4,5,10,11-hexahydro-1H-dibenzo[b,e]-[1,4]diazepin-1-one,
11-(4-ethoxyphenyl)-3-phenyl-2,3,4,5,10,11-hexahydro-1H-dibenzo[b,e]-[1,4]diazepin-1-one,
11-(5-bromo-2-fluorophenyl)-3-phenyl-2,3,4,5,10,11-hexahydro-1H-dibenzo[b,e][1,4]diazepin-1-one, and
11-(2-butoxyphenyl)-3-(3,4-dimethoxyphenyl)-2,3,4,5,10,11-hexahydro-1H-dibenzo[b,e][1,4]diazepin-1-one.

53. The method of claim 42 wherein the composition comprises a compound of Formula I 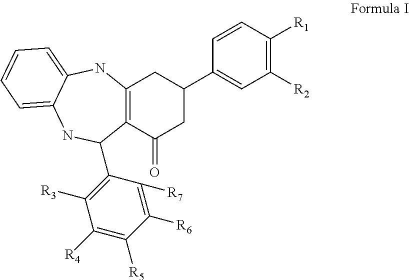
or a pharmaceutically acceptable salt thereof,
wherein:
R₁ and R₂ is each independently selected from the group consisting of hydrogen, alkyl, aryl, heteroaryl, alkoxy, aryloxy, and heteroaryloxy; and
R₃-R₇ is each independently selected from the group consisting of hydrogen, alkyl, aryl, heteroaryl, alkoxy, aryloxy, heteroaryloxy, halo, and haloalkyl.

54. The method of claim 53 wherein the compound is selected from the group consisting of
3-(3,4-dimethoxyphenyl)-11-(3-phenoxyphenyl)-2,3,4,5,10,11-hexahydro-1H-dibenzo[b,e][1,4]diazepin-1-one,
3-(3,4-dimethoxyphenyl)-11-[2-(trifluoromethyl)phenyl]-2,3,4,5,10,11-hexahydro-1H-dibenzo[b,e][1,4]diazepin-1-one,
3-(3,4-dimethoxyphenyl)-11-[3-(trifluoromethyl)phenyl]-2,3,4,5,10,11-hexahydro-1H-dibenzo[b,e][1,4]diazepin-1-one,
3-(3,4-dimethoxyphenyl)-11-(4-propoxyphenyl)-2,3,4,5,10,11-hexahydro-1H-dibenzo[b,e][1,4]-diazepin-1-one,
11-(3-bromophenyl)-3-(3,4-dimethoxyphenyl)-2,3,4,5,10,11-hexahydro-1H-dibenzo[b,e][1,4]diazepin-1-one,
11-(2,5-dimethylphenyl)-3-(4-methoxyphenyl)-2,3,4,5,10,11-hexahydro-1H-dibenzo[b,e][1,4]diazepin-1-one,
11-(3-chloro-5-ethoxy-4-hydroxyphenyl)-3-(4-methoxyphenyl)-2,3,4,5,10,11-hexahydro-1H-dibenzo[b,e][1,4]diazepin-1-one,
11-[4-(methyl-thio)phenyl]-3-phenyl-2,3,4,5,10,11-hexahydro-1H-dibenzo[b,e][1,4]diazepin-1-one,
11-(2-chloro-6-fluorophenyl)-3-phenyl-2,3,4,5,10,11-hexahydro-1H-dibenzo[b,e][1,4]diazepin-1-one, and
11-(3-ethoxyphenyl)-3-(4-methoxyphenyl)-2,3,4,5,10,11-hexahydro-1H-dibenzo[b,e][1,4]-diazepin-1-one.

54. The method of claim 53 wherein the composition comprises a compound of Formula II-1 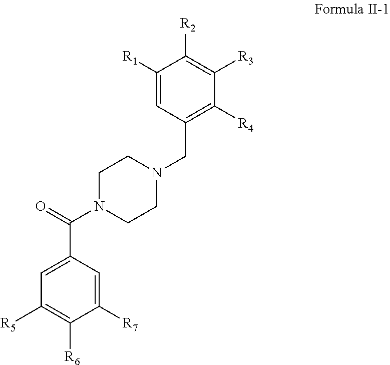
or a pharmaceutically acceptable salt thereof,
wherein R₁-R₇ is each independently selected from the group consisting of hydrogen, alkyl, aryl, heteroaryl, alkoxy, aryloxy, heteroaryloxy, halo, and haloalkyl.

55. The method of claim 54 wherein the compound of claim 34 wherein the compound is selected from the group consisting of
1-(4-biphenylylcarbonyl)-4-(3-methoxybenzyl)piperazine,
1-(4-biphenylylcarbonyl)-4-(2,4-dimethoxybenzyl)piperazine,
1-(4-biphenylylcarbonyl)-4-(5-bromo-2-methoxybenzyl)-piperazine oxalate,
1-(4-biphenylylcarbonyl)-4-(2,3-dimethoxybenzyl)piperazine, and
1-(4-biphenylylcarbonyl)-4-(2,5-dimethoxybenzyl)piperazine.

56. The method of claim 42 wherein the composition comprises a compound of Formula II-2 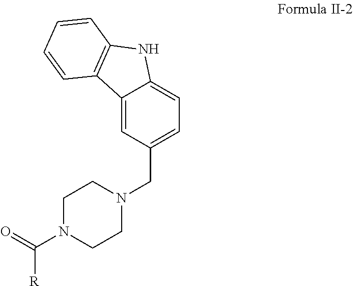
or a pharmaceutically acceptable salt thereof,
wherein R is selected from the group consisting of hydrogen, alkyl, aryl, heteroaryl, alkoxy, aryloxy, heteroaryloxy, halo, and haloalkyl.

57. The method of claim 56 wherein the compound is selected from the group consisting of
4-{[4-[(2-bromo-4-methylphenyl)amino]-6-(4-morpholinyl)-1,3,5-triazin-2-yl]oxy}-3-methoxybenzaldehyde semicarbazone,
3-{[4-(3,4-dimethoxybenzoyl)-1-piperazinyl]methyl}-9-ethyl-9H-carbazole oxalate,
9-ethyl-3-{[4-(4-nitrobenzoyl)-1-piperazinyl]methyl}-9H-carbazole, and
9-ethyl-3-{[4-(2-thienylcarbonyl)-1-piperazinyl]methyl}-9H-carbazole oxalate.

58. The method of claim 42 wherein the composition comprises a compound selected from the group consisting of
1-(diphenylacetyl)-4-(2-nitrobenzyl)piperazine oxalate,
11-(4-biphenylyl)-3,3-dimethyl-2,3,4,5,10,11-hexahydro-1H-dibenzo[b,e][1,4]diazepin-1-one,
4-(1-azepanylsulfonyl)-N-(1-ethyl-2-oxo-1,2-dihydrobenzo[cd]indol-6-yl)benzamide,
1-[3-(2-furyl)acryloyl]-2-methyl-N-phenyl-1,2,3,4-tetrahydro-4-quinolinamine,
1-(2,6-dichlorophenyl)-3-({[2-(3,4-dimethoxy-phenyl)ethyl]amino}methylene)-1,3-dihydro-2H-indol-2-one,
7-(2-oxo-2-phenylethyl)-3-(2-thienyl)-6,7-dihydro-5H-imidazo[2,1-b][1,3]thiazol-4-ium bromide, and
N-(4-{[2-methoxy-5-(4-oxo-3-phenyl-1,2,3,4-tetrahydro-2-quinazolinyl)benzyl]oxy}phenyl)acetamide.
